(19) United States
(12) Patent Application Publication
(10) Pub. No.: US 2026/0327540 A1
(43) Pub. Date: Oct. 22, 2026

(54) A MODULAR INCUBATOR SYSTEM FOR MONITORING THE MORPHOLOGICAL DEVELOPMENT OF A VIABLE BIOLOGICAL MATERIAL DURING INCUBATION

(71) Applicant: ESCO MEDICAL TECHNOLOGIES, UAB, Kaunas (LT)

(72) Inventors: Ricky Lindgaard Nielsen, Egå (DK); Vytautas Virbickas, Kaunas (LT); Kestutis Platkunas, Kaunas (LT); Mikkel Hjerrild Mikkelsen, Vejle (DK); Kim Bøndergaard, Odder (DK)

(21) Appl. No.: 18/994,689

(22) PCT Filed: Jun. 22, 2023
(86) PCT No.: PCT/EP2023/066959

§ 371 Date: Jan. 15, 2025

(30) Foreign Application Priority Data
Nov. 7, 2022 (DK) PA 2022 01011

(51) Int. Cl. C12M 3/00; C12M 1/00; C12M 1/02; C12M 1/34

(52) U.S. Cl. CPC C12M 23/44; C12M 23/40; C12M 41/14; C12M 41/24; C12M 41/36

(57) ABSTRACT

A modular incubator system (500) for incubating a viable biological material M is disclosed. The modular incubator system comprises one or more modular incubator chambers (300) in combination with a docking station (400). In respect of one or more of said one or more modular incubator chambers (300), said modular incubator chamber (300) comprises a housing (302) having a first end (340) and a second end (342), thereby defining a longitudinal direction X between said first end and said second end; wherein said housing comprises a lid (304) which is being configured to be able to shift between an open configuration allowing access to the interior (306) of said modular incubator chamber and a closed configuration, sealing off access to the interior of said modular incubator chamber. The modular incubator positioning a culture dish (310) with the view to accommodate one or more biological materials M within the housing (302) of said modular incubator chamber (300). The housing (302) of said modular incubator chamber (300) comprises a transparent window (316) for enabling capturing of images of a biological material M being accommodated in the interior thereof, through said transparent window. The docking station (400) comprises one or more docking ports (402) for receiving a housing (302) of one or more of said incubator chambers (300) and in respect of one or more docking ports (402) of said docking station (400), said docking port comprises an image capturing device (408) for capturing an image of the interior (306) of a modular incubator chamber (300), once being docked in said docking port (402). The present invention in its various aspects enables incubation of a viable biological material in a modular incubator chamber while at the same time capturing images of that biological material.

### FIELD OF THE INVENTION

The present invention relates in general to the field of incubation of viable biological materials and in particular to incubators for IVF (in vitro fertilization) procedures.

More specifically, the present invention relates in a first aspect to a modular incubator system for incubating a viable biological material, wherein the modular incubator system comprises one or more modular incubator chambers in combination with a docking station.

In a second aspect the present invention relates to a modular incubator chamber for incubating a viable biological material.

In a third aspect the present invention relates to a docking station for docking one or more modular incubator chambers.

In a fourth aspect the present invention provides a use of a modular incubator system according to the first aspect of the invention for incubating a viable biological material.

In a fifth aspect the present invention provides a use of a modular incubator chamber according to the second aspect of the invention for incubating a viable biological material.

In a sixth aspect the present invention provides a use of a docking station according to the third aspect of the invention for incubating a viable biological material.

In a seventh aspect the present invention provides a method for incubating a viable biological material by using a modular incubator system according to the first aspect of the present invention.

### BACKGROUND OF THE INVENTION

The development of in vitro fertilization (IVF) has for the latest few decades resulted in considerably improved methods and techniques which have enhanced success rates of IVF mediated pregnancies and births.

In vitro fertilization involves capturing a ripened egg from a female ovary, fertilizing the ovary with a spermatozoon, incubating the fertilized egg under a controlled environment and subsequently inserting the fertilized and incubated egg into a female's uterus.

As in vitro fertilization is most commonly used by females or couples which notoriously are having problems in getting pregnant the natural way, thus implying some degree of reduced fertility by the male or female counterpart of the couple, or both, and as in vitro fertilization techniques involves quite expensive procedures, these in vitro fertilization techniques are usually performed in a way that seek to optimize efficiency, especially in view of the fact that frequently more than one insertion of a fertilized egg into the female' uterus will be necessary in order to encounter a successful pregnancy.

Additionally, compared to the natural way of getting pregnant, in respect of a couple wherein an individual is having a generic disease or in case of suspicion of such, an IVF mediated pregnancy may be advantageous.

Accordingly, in order to make the in vitro fertilization techniques efficient, the female is typically provided with a hormone treatment prior to harvesting eggs from her ovary. Such hormone treatment will make the female ovary ovulate not only one egg, but a multitude of eggs at the same time.

In order to increase the chance of a viable and successful pregnancy more than one egg from the same female will accordingly be fertilized and incubated concurrently in an incubator.

Prior art incubators include a compartment which allows for accommodating more than one culture dish comprising the fertilized eggs. Hereby, the fertilized egg or embryo may be accommodated and incubated in an incubator under controlled environmental conditions.

Performing a successful in vitro fertilization and incubation of a fertilized egg is not an easy task. One of the major reasons for the rather low success rate of in vitro fertilizations is the absence of reliable methods for providing and maintaining optimum growth conditions for the embryo, including difficulties in avoiding imposing external and detrimental impact on the embryo.

Some of these prior art incubators comprise a housing having one or more doors for providing access to the interior of the incubator. The interior of the incubator holds one or more culture dishes accommodating the embryos to be cultured. Such incubators may be provided with various regulation means for controlling humidity, temperature and gas composition of the interior of the incubator. Some types of incubators additionally comprise an image capturing device for capturing images of an embryo being incubated.

However, once any manual manipulation steps in respect of the embryo are needed, such as manual inspection or adding or removing or exchanging growth media, the culture dish, housing the embryo, will have to be removed from the incubator and arranged on a laboratory bench for inspection and/or manually performing manipulation various steps. Thereby the embryo will encounter a different environment than the environment that has been provided in the interior of the incubator.

Recently, smaller modular incubators have been introduced in the marked. These modular incubators are configured to be stored in docking ports in a docking station which may provide the controlling of physical and chemical parameters encountered by the embryos being accommodated therein. Once any manual manipulation steps in respect of the embryo is needed, such as manual inspection or adding or removing or exchanging growth media, the modular incubator itself may be removed from the docking port of the docking station and arranged on a laboratory bench for easy access to the embryo. Hereby, the change in environment encountered by the embryo upon transferring the culture dish (in its respective modular incubator) is reduced, compared to the situation in which only the culture dish with its embryo is being moved to the laboratory bench for performing the manual manipulation steps.

However, as these modular incubator systems do not include an image capturing device, either any monitoring of the morphological development during incubation of the embryo must be omitted, or the modular incubator must be moved to a microscope stage on a laboratory bench every time such microscopic image is desired. The former situation implies lack of information regarding the morphological development of the embryo and the latter situation implies subjecting the embryo to excessive physical impact due to moving around the embryo every time a microscopic image is needed.

It has been generally found that even very small deviations from what is considered to be optimum incubation conditions for an embryo being incubated in an incubator may have detrimental effects on the quality of the embryo being incubated and hence may reduce the success rate of a resulting pregnancy. Such deviations include excessively subjecting the embryo for physical impacts encountered while moving around the embryo in its culture dish.

Accordingly, too much moving around of an embryo in a culture dish may represent enhanced risks that the IVF procedure ends in an unsuccessful pregnancy once the embryo will be inserted into a female's uterus at a later stage in the IVF process.

Accordingly, a need persists for improved incubators which allow for performing visual monitoring of an embryo while incubating that embryo under predetermined and optimum environmental conditions and at the same time minimizes the degree of deviations from these predetermined and optimum environmental conditions when conducting manually manipulation steps in respect of the incubated embryo at a laboratory bench.

It is an objective of the present invention to fulfill such need.

### BRIEF DESCRIPTION OF THE INVENTION

These objectives are fulfilled according to the various aspect of the present invention.

Accordingly, the first aspect of the present invention relates to a modular incubator system for incubating a viable biological material, said modular incubator system comprising:
one or more modular incubator chambers in combination with
a docking station;
   wherein in respect of one or more of said one or more modular incubator chambers, said modular incubator chamber comprises a housing having a first end and a second end, thereby defining a longitudinal direction X between said first end and said second end;
   wherein said housing comprises a lid, wherein said lid is being configured to be able to shift between an open configuration allowing access to the interior of said modular incubator chamber and a closed configuration, sealing off access to the interior of said modular incubator chamber;
   wherein said modular incubator chamber, at said interior thereof, comprises a culture dish support for positioning a culture dish with the view to accommodate one or more biological materials M within the housing of said modular incubator chamber;
   wherein said housing of said modular incubator chamber comprises a transparent window for enabling capturing of images of a biological material M being accommodated in the interior thereof, through said transparent window;
   wherein said docking station comprises one or more docking ports for receiving a housing of one or more of said incubator chambers;
   wherein in respect of one or more docking ports of said docking station, said docking port comprises an image capturing device for capturing an image of the interior of a modular incubator chamber, once being docked in said docking port.

In a second aspect the present invention relates to a modular incubator chamber comprising:

a housing having a first end and a second end, thereby defining a longitudinal direction X between said first end and said second end;
wherein said housing comprises a lid, wherein said lid is being configured to be able to shift between an open configuration allowing access to the interior of said modular incubator chamber and a closed configuration, sealing off access to the interior of said modular incubator chamber;
wherein said modular incubator chamber, at said interior thereof, comprises a culture dish support for positioning a culture dish with the view to accommodate one or more biological materials M within the housing of said modular incubator chamber;
wherein said housing of said modular incubator chamber comprises a transparent window for enabling capturing of images of a biological material M being accommodated in the interior thereof, through said transparent window.

In a third aspect the present invention relates to a docking station for docking one or more modular incubator chambers; wherein said docking station comprises one or more docking ports for receiving a housing of one or more of said incubator chambers; wherein in respect of one or more docking ports of said docking station, said docking port comprises an image capturing device for capturing an image of the interior of a modular incubator chamber, once being docked in said docking port.

In a fourth aspect the present invention provides a use of a modular incubator system according to the first aspect of the present invention for incubating a viable biological material.

In a fifth aspect the present invention provides a use of a modular incubator chamber according to the second aspect of the present invention for incubating a viable biological material.

In a sixth aspect the present invention provides a use of a docking station according to the third aspect of the present invention for incubating a viable biological material.

In a seventh aspect the present invention provides method of incubating a viable biological material, wherein said method comprises:
i) providing a modular incubator system according to the first aspect of the present invention;
ii) providing a viable biological material;
iii) arranging said viable biological material in a culture dish and subsequently arranging said culture dish in the interior of a modular incubator chamber of said modular incubator system;
iv) docking said modular incubator chamber in a docking port of said docking station of said incubator system;
v) allowing said viable biological material to be incubated in said modular incubator chamber;
vi) whenever desired, allowing said image capturing device to capture one or more images of said biological material being accommodated in said culture dish.

The present invention in its various aspects allows for enabling image capturing of a viable biological material, such as an oocyte or an embryo during incubation thereof under a predetermined an optimum environment, while at the same time minimizing any detrimental effects caused by deviating from that environment when conducting manually manipulation steps at a laboratory bench in respect of the incubated embryo.

### BRIEF DESCRIPTION OF THE FIGURES

FIG. 1 is a perspective view illustrating the general concept of designing an incubator as an incubator system comprising a plurality of modular incubator chambers in combination with a docking station having a plurality of docking ports.

FIG. 2a is a top perspective view showing a modular incubator chamber of the modular incubator system of the invention.

FIG. 2b is a top plan view showing the modular incubator chamber of FIG. 2a.

FIG. 2c is a plan rear view showing the modular incubator chamber of FIGS. 2a and 2b.

FIG. 3 is a cross-sectional view illustrating the the modular incubator chamber illustrated in FIGS. 2a, 2b and 2c.

FIG. 4 is a perspective cross-sectional view illustrating further details of the the modular incubator chamber of the present invention.

FIGS. 5a and 5b are drawings illustrating the working modes of valves of a valve system to be used with the modular incubator chamber and the associated docking port of the docking station of the docking system of the present invention.

FIG. 6 is a diagram illustrating one embodiment of a design of a gas supply system comprising a gas source and a gas distribution system to be used with the docking station of the modular incubator system of the present invention.

FIG. 7 is a diagram illustrating the concept of a gas source which may be incorporated in the docking station of the modular incubator system of the present invention.

FIG. 8 is a diagram illustrating the working mode of the controlling of the modular incubator system according to the invention.

### DETAILED DESCRIPTION OF THE INVENTION

### The First Aspect of the Present Invention

The fist aspect of the present invention relates to a modular incubator system 500 for incubating a viable biological material M, said modular incubator system comprising:
one or more modular incubator chambers 300 in combination with
a docking station 400;
   wherein in respect of one or more of said one or more modular incubator chambers 300, said modular incubator chamber 300 comprises a housing 302 having a first end 340 and a second end 342, thereby defining a longitudinal direction X between said first end and said second end;
   wherein said housing comprises a lid 304, wherein said lid is being configured to be able to shift between an open configuration allowing access to the interior 306 of said modular incubator chamber and a closed configuration, sealing off access to the interior of said modular incubator chamber;
   wherein said modular incubator chamber 300, at said interior 306 thereof, comprises a culture dish support 308 for positioning a culture dish 310 with the view to accommodate one or more biological materials M within the housing 302 of said modular incubator chamber 300;
   wherein said housing 302 of said modular incubator chamber 300 comprises a transparent window 316 for enabling capturing of images of a biological material M being accommodated in the interior thereof, through said transparent window;
   wherein said docking station 400 comprises one or more docking ports 402 for receiving a housing 302 of one or more of said incubator chambers 300;
   wherein in respect of one or more docking ports 402 of said docking station 400, said docking port comprises an image capturing device 408 for capturing an image of the interior 306 of a modular incubator chamber 300, once being docked in said docking port 402.

Accordingly, the present invention relates in its first aspect to a modular incubator system 500 comprising one or more modular incubator chambers 300 in combination with a docking station 400. The incubator chambers 300 and the docking station 400 are configured in such a way that the modular incubator chambers 300 can be docked in a docking port 402 and in such a way that an image capturing device 408 of the docking port will be able to capture images of a viable biological material being accommodated within the interior 306 of the modular incubator chamber 300 while being incubated therein and while the modular incubator chamber 300 is docked in that docking port 402.

The capturing of the images is performed through a transparent window 316 in the housing 302 of the modular incubator chamber 300.

The modular incubator chamber 300 itself allows for upholding a desired and predetermined and optimum environment in respect of the embryo being incubated, such as a desired and predetermined and optimum gaseous atmosphere, even in a situation where the modular incubator chamber is being removed from its respective docking port 402 of the docking station 400 of the modular incubator system 500.

Hereby, a predetermined and optimum environment which to a larger degree mimics the environment of a fallopian tube or a uterus of a female is obtained when a viable biological material in its modular incubator chamber 300 is being incubated and moved between the associated docking port 402 of the docking station 400 and a laboratory bench with the view to perform the necessary manipulation steps.

In the present invention the term “modular incubator system” shall be construed to mean a system comprising a docking station in combination with one or more incubator chambers, wherein the one or more incubator chambers is/are configured to be docked in respective docking ports of that docking station. The modular incubator system is intended for incubation or cultivation of a viable biological material.

The incubator system comprising the docking station and one or more incubator chamber(s) in general is configured for providing some kind of interaction between the docking station and the incubator chambers being docked therein.

Such interactions may be one or more of the following: providing a gas having a desired composition to the incubator chamber(s); providing electricity to the incubator chamber(s) for powering hearing elements thereof and/or for powering a light source in the incubator chamber(s); allowing monitoring of the viable biological material being present in the incubator chamber(s), such as by means of an image capturing device which is located in the docking station.

In should be understood that within the meaning of the present application, the term “modular incubator system” shall be construed in such a way that the incubator chambers are configured to be used for incubation of a viable biological material, irrespective of whether the individual incubator chamber is being docked in a docking port of the docking station, or whether that incubator chamber is removed from the docking port of the docking station.

In this way, it is to be understood that cultivation or incubation of a viable biological material of the individual incubator chambers may take place and/or be continued even after that incubator chamber has been removed from its docking station and placed e.g. on a laboratory bench. Hereby manual manipulation operations, such as shift or control of culture or growth media, manual inspection by use of a laboratory microscope or the like can take place. Such operation are preferably carried out under a hood providing a desired gas atmosphere.

In preferred embodiment, and in order to make such manual manipulation operations practical conceivable, when the individual incubation chamber has been removed from a docking port, the incubation chamber is configured in a way that enables support on a planar, horizontal support surface. This may be attained by providing the bottom part of the incubator chamber with one or more supports or simply by making the bottom part of the incubator chamber comprise a flat surface.

In preferred embodiments the incubation chamber, in the orientation intended during use for incubation, is having its maximum dimension in a horizontal direction.

In this way the dimension of the incubation chamber in a horizontal direction is greater than the dimension in a vertical direction. Hereby, adequate stability is attended when the incubator chamber is used for incubation at a location outside a docking port of the docking station.

The individual incubator chambers may in embodiments comprise a display, such as an electronic display, for providing information relating to the identity of the viable biological material being accommodated in the incubator chamber.

It should be understood that in some embodiments the present invention does not relate to methods or uses which involve treatment of the human or animal body by surgery or diagnostic methods practiced on the human or animal body.

It should also be understood that that in other embodiments the present invention may relate to methods or uses which involve treatment of the human or animal body by surgery or diagnostic methods practiced on the human or animal body.

In an embodiment of the modular incubator system 500 according to the first aspect of the present invention and in respect of one or more of said one or more modular incubator chambers 300 and in respect of one or more of said one or more docking ports 402 of said docking station 400, the position of said transparent window 316 of said modular incubator chamber 300 is adapted to the position of said image capturing device 408 in said docking port 402 in a way that enables capturing of images by said image capturing device 408 through said transparent window 316 of said modular incubator chamber 300, once said modular incubator chamber 300 is being docked in said docking port 402.

Hereby is ensured that the image capturing device 408 will be able to capture images of the interior 306 of that modular incubator chamber 300 through that transparent window 316.

In an embodiment of the modular incubator system 500 according to the first aspect of the present invention and in respect of one or more of said one or more modular incubator chambers 300, said transparent window 316 of said modular incubator chamber 300 is arranged at a bottom part 358 of said housing.

As it is preferred to arrange the image capturing device 408 at a position in the docking port 402 of the docking station where it will focus in an upward direction, the transparent window 316 of the modular incubator chamber 300 is conveniently arranged at a bottom part 358 of the housing 302 of said modular incubator chamber 300.

In an embodiment of the modular incubator system 500 according to the first aspect of the present invention and in respect of one or more of said one or more modular incubator chambers 300, said culture dish support 308 of said modular incubator chamber 300 is being arranged above said transparent window 316.

Hereby is assured that when the image capturing device 408 focuses in a focus direction corresponding to said transparent window 316, that image capturing device 408 will focus on the area of a culture dish 410 being arranged on said culture dish support 308.

In an embodiment of the modular incubator system 500 according to the first aspect of the present invention and in respect of one or more of said one or more modular incubator chambers 300, said transparent window 316 of said housing 302 of said modular incubator chamber is having an elongate shape, such as an elongate and linear extension extending in a direction Y, transversal to said longitudinal direction X of said housing of said modular incubation chamber 300.

Hereby the image capturing device 408 will be able to capture images of a multitude of viable biological materials which are being arranged in-line in a culture dish 310 in the interior 306 of the modular incubator chamber 300.

In an embodiment of the modular incubator system 500 according to the first aspect of the present invention and in respect of one or more of said one or more modular incubator chambers 300 and in respect of one or more of said one or more docking ports 402 of said docking station 400, said modular incubator chamber 300 is being configured to be docked in said docking port 402 with its first or rear end 340 facing said docking port 402.

In an embodiment of the modular incubator system 500 according to the first aspect of the present invention and in respect of one or more of said one or more modular incubator chambers 300, said modular incubator chamber 300, in the interior 306 thereof, comprises a light source 372 for directing light to the area of the culture dish support 308 of said modular incubator chamber 300, thereby enabling illumination of a viable biological material in a situation of capturing images of said viable biological material.

The light source will improve the quality of images captured by the image capturing device 408 in an image capturing situation.

In an embodiment said light source 372 is being attached to said lid 304 of the housing 302 of said modular incubator chamber 300, at an inner side thereof.

Hereby light can easily be directed to a viable biological material which is arranged at a lower part of the interior 306 of the modular incubator chamber 300.

In an embodiment said light source 372 is being selected from the group of one or more LEDs, one or more laser diodes, one or more incandescent light bulbs.

It is to be understood, that in order not to subject the viable biological material being incubated to excessive disturbances, the light source 372 should only be switched on in those short periods of time in which the image capturing unit 408 is in fact in the process of capturing an image.

In an embodiment of the modular incubator system 500 according to the first aspect of the present invention and in respect of one or more of said one or more modular incubator chambers 300, said culture dish support 308 is defining a planar support surface for supporting said culture dish 310.

In an embodiment of the modular incubator system 500 according to the first aspect of the present invention and in respect of one or more of said one or more modular incubator chambers 300, said housing 302 of said modular incubator chamber 300, such as at an outer portion thereof, is being provided with electric connectors 322 for providing electric power and/or electric signals to said modular incubator chamber; and wherein in respect of one or more docking ports 402 of said docking station 400, said docking port is being provided with electric connectors 410, thereby allowing providing electric power and/or electric signals between said docking port 402 of said docking station 400 and a modular incubator chamber 300 being docked therein.

Hereby conveying of electric power or electric signals between said docking port 402 and a modular incubator chamber 300 is enabled.

In an embodiment of the modular incubator system 500 according to the first aspect of the present invention and in respect of one or more of said one or more modular incubator chambers 300, said lid 304 is being a hinged lid which is being connected to said housing of said modular incubator chamber via a hinge.

In an embodiment of the modular incubator system 500 according to the first aspect of the present invention and in respect of one or more of said one or more modular incubator chambers 300, said housing 302 of said modular incubator chamber 300 comprises a display 324 which is being configured to display information relating to an operational status of the incubation taking place in said modular incubator chamber.

In an embodiment of the modular incubator system 500 according to the first aspect of the present invention said image capturing device 408 comprises microscopic optics so as to enable capturing of microscope images.

Hereby magnified images may be captured which improves study of the morphological nature of the biological materials being incubated.

In an embodiment of the modular incubator system 500 according to the first aspect of the present invention the number of modular incubator chambers 300 of said modular incubator system 500 is selected from the ranges 1-100, such as 2-95, for example 5-90, e.g. 10-85, such as 15-80, for example 20-75, e.g. 25-70, 30-65, such as 35-60, e.g. 40-55 or 45-50.

In an embodiment of the modular incubator system 500 according to the first aspect of the present invention the number of docking ports 402 in said docking station 400 of said modular incubator system 500 is selected from the ranges 1-100, such as 2-95, for example 5-90, e.g. 10-85, such as 15-80, for example 20-75, e.g. 25-70, 30-65, such as 35-60, e.g. 40-55 or 45-50.

In an embodiment of the modular incubator system 500 according to the first aspect of the present invention the docking station 400 comprises said docking ports 402 in an arrangement of one or more shelves of adjacently positioned docking ports 402, wherein in case said docking station comprises two or more shelves, said shelves are being arranged above each other.

In an embodiment of the modular incubator system 500 according to the first aspect of the present invention in respect of one or more of said one or more modular incubator chambers 300, said modular incubator chamber comprises an incubation chamber engagement means 326 and wherein in respect of one or more docking ports 402 of said docking station 400, said docking port comprises a docking port engagement means 414, wherein said incubation chamber engagement means 326 is being configured to enter into engagement with said docking port engagement means 414 so as to provide ease and proper positioning and optionally also fixing said modular incubator chamber 300 in said docking port 402, as well as detaching said modular incubator chamber 300 from said docking port 402 of said docking station 400.

Hereby, easy and proper positioning and optionally also fixing said modular incubator chamber 300 in said docking port 402, as well as detaching said modular incubator chamber 300 from said docking port 402 of said docking station 400 is provided.

In an embodiment of the modular incubator system 500 according to the first aspect of the present invention the modular incubator system 500 comprises an image processing unit 660 for image processing of images captured by said image capturing device 408, wherein said modular incubator system 500 optionally furthermore comprises a data storage 658 for storing images captured by said image capturing units 408 and/or for storing images processed by said image processing unit 660.

An image processing unit is beneficial for manipulating the images captured, such as for adjusting contrast, for filtering and for generating time-lapse series of images.

In an embodiment one or more of said image capturing devices 408 of said docking ports 402 of said docking station is/are being coupled to an image processing unit 660.

In an embodiment of the modular incubator system 500 according to the first aspect of the present invention and in respect of one or more specific docking ports 402 of said docking station 400, said specific docking port comprises its own dedicated image capturing device 408 which is configured to only capture images relating to a modular incubator chamber 300 which is being docked in said specific docking port 402.

In another embodiment of the modular incubator system 500 according to the first aspect of the present invention and respect of a number N of adjacently arranged docking ports 402 of said docking station 400, said adjacently arranged docking ports share a common image capturing device 408 in the sense that one and only one image capturing device is responsible for capturing images relating to a modular incubator chamber 300 which is being docked in one of said N adjacently arranged docking ports 402, wherein said docking station comprises a displacement device 482 for enabling displacement of said common image capturing device 408 in relation to said N adjacently arranged docking ports 402 of said docking station 400.

Hereby one image capturing device is responsible for the capturing of images of biological materials being accommodated in different modular incubator chambers which are being docked in different docking port 402 of the docking station 400.

In an embodiment the number N is being an integer selected in the ranges of 2-25 or more, such as 4-22, for example 6-20, such as 8-18, such as 10-16 or 12-14.

Independently, one or more image capturing devices 408, preferably all image capturing devices 408 of the docking station 400 may comprise or be coupled to a displacement device 482, such as an electrically driven and remotely controlled displacement device 482 for enabling displacement of said common image capturing device 408 in a direction transversal to the longitudinal direction X of a modular incubator chamber 300 being docked in a docking port 402 with the view to enable such capturing device 408 to focus on more than one culture well in a culture dish 310 being accommodated in the interior of the modular incubator chamber 300, wherein such culture wells are arranged in such direction transversal to the longitudinal direction X.

In an embodiment of the modular incubator system 500 according to the first aspect of the present invention and in respect of one or more of said modular incubator chambers 300, said modular incubator chamber comprises in its interior 306 an electric heating element 318 for heating the interior of said modular incubator chamber, and wherein said modular incubator chamber comprises a power source 320 for providing power to said heating element 318, wherein said electric heating element 318 is being electrically connected to said power source 320.

In an embodiment said power source 320 is being an electric power source, such as a battery, for example a rechargeable battery.

In an embodiment said heating element 318 is being thermally connected to a heat distribution element for distributing heat dissipated in said heating element; wherein said heat distribution element is being arranged, at least partly, in the interior 306 of said modular incubator chamber 300.

In an embodiment the chamber comprises a thermostat 374 and an electric thermostatic circuit 376, wherein said electric heating element 318, said power source 320 and said thermostat 374 are being electrically connected in said electric thermostatic circuit 376 so as to enable thermostatic control of the temperature inside said modular incubator chamber 300.

The above embodiments provide for upholding a desirable and predetermined and optionally also optimum temperature in the interior 306 of the modular incubator chamber 300 in a situation where the modular incubator chamber is removed from its associated docking port 402 with the view to perform visual inspection and manual replenishing, removal or exchange of growth medium to the biological materials being incubated.

In an embodiment of the modular incubator system 500 according to the first aspect of the present invention and in respect of one or more of said one or more modular incubator chambers 300, said modular incubator chamber 300 comprises a chamber inlet opening for gas 312, wherein said chamber inlet opening for gas 312 is being in fluid connection with the interior 306 of said modular incubator chamber; and wherein said modular incubator chamber 300 furthermore comprises a chamber outlet opening for gas 314, wherein said chamber outlet opening for gas 314 is being in fluid connection with the interior 306 of said modular incubator chamber; and wherein in respect of one or more docking ports 402 of said docking station 400, said docking port 402 comprises a docking port outlet opening for gas 404 and a docking port inlet opening for gas 406; thereby enabling transfer of gas from said docking port 402 of said docking station 400 to the interior 306 of said modular incubator chamber 300 via said docking port outlet opening for gas 404 and said chamber inlet opening for gas 312; and thereby enabling transfer of gas from the interior 306 of said modular incubator chamber 300 to said docking port 402 of said docking station 400 via said chamber outlet opening for gas 314 and said docking port inlet opening for gas 406.

In an embodiment of the modular incubator system 500 according to the first aspect of the present invention and in respect of one or more of said one or more modular incubator chambers 300, and in respect of one or more of said one or more docking ports 402 of said docking station 400, the position of said chamber inlet opening for gas 312 of said housing 302 of said modular incubator chamber 300 and the position of said docking port outlet opening for gas 404 of said docking port 402 are adapted to each other in such a way that once docking said modular incubator chamber 300 in said docking port 402, said chamber inlet opening for gas 312 of said housing 302 of said modular incubator chamber 300 and said docking port outlet opening for gas 404 of said docking port 402 will be in fluid connection, thereby enabling transfer of gas from said docking port 402 to said modular incubator chamber 300; and wherein the position of said chamber outlet opening for gas 314 of said housing 302 of said modular incubator chamber 300 and the position of said docking port inlet opening for gas 406 of said docking port 402 are adapted to each other in such a way that once docking said modular incubator chamber 300 in said docking port 402, said chamber outlet opening for gas 314 of said housing 302 of said modular incubator chamber 300 and said docking port inlet opening for gas 406 of said docking port 402 will be in fluid connection, thereby enabling transfer of gas from said modular incubator chamber 300 to said docking port 402.

These embodiments ensure that gas having a desired composition can be delivered from a gas source 202 via a gas distribution system 204 to the interior 306 of the modular incubator chamber 300 via the docking port outlet opening for gas 404 and the chamber inlet opening for gas 312, and gas from the interior 306 of the modular incubator chamber 300 can be returned to the gas source 202 via the chamber outlet opening for gas 314 and the docking port inlet opening for gas 406.

In an embodiment of the modular incubator system 500 according to the first aspect of the present invention the docking port outlet opening for gas 404 of said docking port 402 comprises a valve 4 and said chamber inlet opening for gas 312 of said housing 302 comprises a valve 2; and said chamber outlet opening for gas 314 comprises a valve 2 and said docking port inlet opening for gas 406 of said docking port 402 comprises a valve 4.

Hereby can be assured that gas will only flow into the docking port 402 once a modular incubator chamber 300 is being arranged in that docking port 402. In other words, no gas will flow into the docking port 402 unless a modular incubator chamber 300 is being docked therein. Moreover, this embodiment assures that once a modular incubator chamber 300 is being removed form a docking port, no atmospheric air will enter through the chamber inlet opening for gas 312 and the chamber outlet opening for gas 314 of that chamber 300.

Accordingly, there will be no contamination of the gas atmosphere being present in the interior 306 of the modular incubator chamber 300, once that chamber 300 is being removed from its docking port 402.

In an embodiment of the modular incubator system 500 according to the first aspect of the present invention and in respect of one or more of said one or more modular incubator chambers 300, said valve 2 of said chamber inlet opening for gas 312 and said valve 2 of said chamber outlet opening for gas 314 each comprises a valve body 6 having a front end 10, a rear end 12 and a through-going channel 14 therein, and a spring-loaded displaceable valve element 8, wherein said displaceable valve element 8 is being arranged in said through-going channel 14; wherein said displaceable valve element 8 is being configured to be displaceable in said through-going channel 14 of said valve body 6 in such a way, that when not acted upon by an external force, said spring-loaded displaceable valve element 8 is not being displaced in said through-going channel 14 of said valve body 6, thereby making said valve attain a closed configuration blocking passage of gas through said through-going channel 14, and in such a way, that when acted upon by an external force, said spring-loaded displaceable valve element 8 is being displaced in said through-going channel 14 of said valve body 6, thereby making said valve 2 attain an open configuration, allowing passage of gas through said through-going channel 14; and
in in respect of one or more of said one or more docking ports 402 of said docking station 400, said valve 4 of said docking port outlet opening for gas 404 and said valve 4 of said docking port inlet opening for gas 406 each comprises a valve body 16 having a front end 20, a rear end 22 and a through-going channel 24 therein, and a spring-loaded displaceable valve element 18, wherein said displaceable valve element 18 is being arranged in said through-going channel 24; wherein said displaceable valve element 18 is being configured to be displaceable in said through-going channel 24 of said valve body 16 in such a way, that when not acted upon by an external force, said spring-loaded displaceable valve element 18 is not being displaced in said through-going channel 24 of said valve body 16, thereby making said valve attain a closed configuration, blocking passage of gas through said through-going channel 24, and in such a way, that when acted upon by an external force, said spring-loaded displaceable valve element 18 is being displaced in said through-going channel 24 of said valve body 16, thereby making said valve 4 attain an open configuration, allowing passage of gas through said through-going channel 24.

Hereby, each of the two valves 2,4 will be able to change configuration between an open and a closed configuration by displacement of the respective valve element 8,18 in the associated valve body 6,16.

In an embodiment of the modular incubator system 500 according to the first aspect of the present invention and in respect of one or more of said one or more docking ports 402 of said docking station 400, and in respect of one or more of said one or more modular incubator chambers 300 said valves 2,4 are having dimensions and geometries in such a way that once docking said modular incubator chamber 300 in said docking port 402 of said docking station 400, said displaceable valve element 8 of said valve 2 and said displaceable valve element 18 of said valve 4 will displace each other into their respective valve bodies 6,16, thereby opening said valves 2,4 of said docking port outlet opening for gas 404 and said chamber inlet opening for gas 312; and thereby opening said valves 2,4 of said chamber outlet opening for gas 314 and said docking port inlet opening for gas 406.

In an embodiment of the modular incubator system 500 according to the first aspect of the present invention and in respect of one or more of said docking ports 402 of said docking station 400 of said modular incubator system 500, preferably in respect of all said docking ports 402, said docking port outlet opening for gas 404 comprises a flow restrictor for restricting the magnitude of flow of gas flowing into said docking port 402.

In one embodiment the flow restrictor may comprises a tube through which the gas is conveyed to said docking port 402, wherein said tube optionally is having a cross-sectional area selected from the ranges of 0.2-8 mm², such as 0.5-7 mm², for example 1-6 mm², such as 2-5 mm² or 3-4 mm²; and/or the length of said tube is optionally selected from the ranges of 5-30 mm, such as 8-25 mm, for example 10-22 mm, e.g. 15-20 mm.

Such a flow restrictor aids in balancing the flow of gas through the docking ports 402 comprising a modular incubator chamber 300 with the capacity of the gas supply system 200 and thereby also aids in making the flow of gas through the different docking ports 402 equal to each other.

Hereby, each of the two valves 2,4 will open the other valve 4,2 once being brought into contact with each other by making their respective front ends 10,20 meet.

In an embodiment of the modular incubator system 500 according to the first aspect of the present invention the docking station 400 comprises a gas distribution system 204 for supplying gas to and from one or more of said one or more docking ports 402, wherein said gas distribution system 204 comprises a main gas supply line 210 and a main gas return line 212, wherein in respect of one or more of said docking ports 402, said docking port inlet opening for gas 404 is being fluidly connected to said main gas supply line 210, and said docking port outlet opening for gas 406 is being fluidly connected to said main gas return line 212.

In one embodiment said gas distribution system 204 comprises a number of manifold pairs 214, wherein each manifold pair comprises an inlet manifold 216 and an outlet manifold 218, wherein said inlet manifold 216 is being fluidly connected to said main gas supply line 210 and wherein said outlet manifold 218 is being fluidly connected to said main gas return line 212; wherein each manifold pair 214 is connected to one or more docking ports 402 of said docking station 400 in such a way that in respect of a specific manifold pair 214, and in respect of said one or more docking ports 402 being connected thereto, said docking port outlet opening for gas 404 of said docking port 402 is being fluidly connected to said inlet manifold 216, and said docking port inlet opening for gas 406 of said docking port 402 is being fluidly connected to said outlet manifold 218.

In one embodiment said docking station 400 comprises a gas supply system 200, wherein said gas supply system 200 comprises a gas source 202 and said gas distribution system 204, wherein said gas source comprises a supply gas outlet 206 and a return gas inlet 208, wherein said supply gas outlet 206 of said gas source 202 is being fluidly connected to said main gas supply line 210 of said gas distribution system 204, and wherein said return gas inlet 208 of said gas source 202 is being fluidly connected to said main gas return line 212 of said gas distribution system 204.

In these embodiments comprising a gas distribution system 204 it is possible to supply gas from a gas source 202 to the docking ports 402 via the main gas supply line 210 and to return gas from the docking ports to the gas source 202 via the main gas return line 212.

In an embodiment of the modular incubator system 500 according to the first aspect of the present invention, the gas source 202 of said gas supply system 200 comprises a gas mixing box 242 fluidly connected to said supply gas outlet 206 and said return gas inlet 208 of said gas source, wherein said main gas supply line 210 of said gas distribution system 204 is being fluidly connected to said supply gas outlet 206, and wherein said main gas return line 212 of said gas distribution system 204 is being fluidly connected to said return gas inlet 208 of said gas source 202, thereby forming a flow loop 244 comprising said gas distribution system 204 and said gas mixing box 242; wherein said flow loop comprises a pump 246.

Hereby circulating gas in said loop, and also through the gas distribution system 204 of the docking station 400 is possible.

The purpose of the gas source is to provide and deliver a desired gas composition to the gas distribution system 204 including the various docking ports 402 of the docking station 400 with the view to supply this gas to the interior 306 of the modular incubator chambers 300.

In one embodiment of this embodiment the pump 246 is being arranged downstream in relation to said main gas return line 212.

In one embodiment the flow loop 244 comprises a pump oscillation damper 247, wherein said pump oscillation damper optionally is being arranged immediately downstream in relation to said pump 246.

The pump oscillation damper will equalize small and rapid pressure variations caused by each pump stroke of the pump.

In an embodiment of the modular incubator system 500 according to the first aspect of the present invention, the flow loop 244 comprises a pressure sensor, such as a differential pressure sensor 248 for sensing the pressure of gas supplied to said main gas supply line 210 of said gas distribution system 204, wherein said pressure senor 248 optionally is being arranged immediately upstream in relation to said main gas supply line 210 of said gas distribution system 204.

The pressure sensor 248 allows for regulating the pump 246 in order to maintain a desired pressure in the flow loop 244 by feedback.

In one embodiment the pressure sensor 248 is being a differential pressure sensor, sensing a pressure relative to the pressure of the return gas inlet 208.

In one embodiment the flow loop 244 comprises a release valve 249 for enabling pressure relief in said flow loop, wherein said release valve optionally is being arranged immediately downstream in relation to said main gas return line 212 of said gas distribution system 402.

The pressure release valve 249 enables improved control of the pressure in the flow loop 344.

In an embodiment of the modular incubator system 500 according to the first aspect of the present invention, the gas mixing box 242 comprises an inlet for N₂ gas 250; and an inlet for CO₂ gas 251, wherein said inlet for N₂ gas 250 is fluidly connected to an N₂ valve 252 for regulating the inflow of N2, and an N₂ mass flow sensor 253 arranged downstream of said N₂ valve 252 for sensing the amount of N₂ flowing into said gas mixing box 242; and wherein said inlet for CO₂ gas 251 is fluidly connected to a CO₂ valve 254 for regulating the inflow of CO₂, and an CO₂ mass flow sensor 255 arranged downstream of said CO₂ valve 254 for sensing the amount of CO₂ flowing into said gas mixing box 242.

Herby it is possible to control the inlet of N₂ gas and the inlet of CO₂ gas into the gas mixing box 242 with the view to obtain a desired, predetermined and optimum gas composition in the gas mixing box 242.

In an embodiment of the modular incubator system 500 according to the first aspect of the present invention, the flow loop 244 comprises a mass flow sensor 256 arranged at an upstream position in relation to said gas mixing box 242 for sensing the amount of return gas entering said gas mixing box.

Information relating to the amount of return gas entering said gas mixing box is used for determining the total amount of N₂ gas and CO₂ gas which needs to be introduced into the gas mixing box 242.

In an embodiment of the modular incubator system 500 according to the first aspect of the present invention, the gas source 202 comprises an O₂ sensor 258 for sensing the concentration of O₂ exiting said gas distribution system 204; and wherein said gas source 202 comprises a CO₂ sensor 260 for sensing the concentration of CO₂ exiting said gas distribution system 204, wherein said O₂ sensor and/or said CO₂ sensor optionally is/are being arranged downstream in relation to said pump 246.

Information relating to the concentration of O₂ and the concentration of CO₂ exiting said gas distribution system 204 is used for determining the specific amount of N₂ gas and the specific amount of CO₂ gas which needs to be introduced into the gas mixing box 242.

In an embodiment of the modular incubator system 500 according to the first aspect of the present invention, the gas source 202 comprises a temperature sensor 262 for sensing the temperature of gas circulating in said flow loop 244, wherein said temperature sensor optionally is being arranged downstream in relation to said pump 246, preferably at a position corresponding to the position of said O₂ sensor 258.

In an embodiment of the modular incubator system 500 according to the first aspect of the present invention, the gas source 202 comprises a pressure sensor 264 for sensing the absolute pressure in said flow loop 244 wherein said pressure sensor optionally is being arranged downstream in relation to said pump 246, preferably at a position corresponding to the position of said CO₂ sensor 260.

The temperature sensor 262 and pressure sensor 264 are useful for performing compensation of the readings of the O₂ sensor 258 due to temperature sensitivity thereof and the readings of the CO₂ sensor 260 due to sensitivity thereof towards pressure.

In an embodiment of the modular incubator system according to the first aspect of the present invention, the flow loop 244 comprises a UV sanitizer 266 for sanitizing gas flowing in said flow loop 244 via electromagnetic radiation in the UV range, wherein said UV sanitizer optionally being arranged immediately downstream in relation to said main gas return line 212.

In an embodiment of the modular incubator system 500 according to the first aspect of the present invention, the gas source 202 comprises one or more filters 268, such as HEPA and/or VOCs filters, wherein such a filter is being arranged immediately upstream in relation to said main gas supply line 210, and/or wherein such a filter is being arranged immediately upstream in relation to the inlet for N₂ gas 250 into said gas mixing box 242; and/or wherein such a filter is being arranged immediately upstream in relation to the inlet for CO₂ gas 251 into said gas mixing box 242.

In an embodiment of the modular incubator system 500 according to the first aspect of the present invention, the gas source 202 comprises a gas mixing control system 270, wherein said gas mixing control system is electrically connected to one or more of the following sensors for receiving sensing signals therefrom: said N₂ mass flow sensor 253 for sensing the amount of N₂ flowing into said gas mixing box; said CO₂ mass flow sensor 255 for sensing the amount of CO₂ flowing into said gas mixing box; said mass flow sensor 256 for sensing the amount of return gas entering said gas mixing box; said O₂ sensor 258 for sensing the concentration of O₂ exiting said main gas return line 212 of said gas distribution system 204; said CO₂ sensor 260 for sensing the concentration of CO₂ exiting said main gas return line 212 of said gas distribution system 204; said temperature sensor 262 for sensing the temperature circulating in said flow loop 244; said pressure sensor 264 for sensing an absolute pressure in said flow loop 244, said pressure sensor 248 for sensing the pressure of gas supplied to said gas main gas supply line 210 of said distribution system 204.

This embodiment enables gaining information of various parameters which are to be used in providing a feed back when controlling the operation of the gas source 202.

In an embodiment of the modular incubator system 500 according to the first aspect of the present invention, the gas mixing control system 270 is electrically connected to one or more of the following elements for control thereof: said N₂ valve 252 for regulating the inflow of N₂ into said gas mixing box 242; said CO₂ valve 254 for regulating the inflow of CO₂ to said gas mixing box 242; said pump 246 for circulating gas in said flow loop 244; said release valve 249.

This embodiment enables providing a feed back when controlling the operation of the gas source 202.

In one embodiment the gas mixing control system 270 is being configured to receive input from said pressure sensor 248 and on the basis thereof control said pump 246, optionally also to activate said release valve 249 in order to maintain a desired and predetermined pressure of gas supplied to said main gas supply line 210 of said gas distribution system 204.

Hereby the pressure in the flow loop 244 can be controlled.

In an embodiment of the modular incubator system 500 according to the first aspect of the present invention, the gas mixing control system 270 is being configured to receive input from said mass flow sensor 256, and on the basis on said input to determine the total amount of CO₂ gas and N₂ gas needed to be supplied via said inlet for CO₂ gas 251 and via said inlet for N₂ gas 250 according to desired and predetermined criteria.

In an embodiment of the modular incubator system 500 according to the first aspect of the present invention, the gas mixing control system 270 is being configured to receive input from said CO₂ sensor 260 and said O₂ sensor 258, and on the basis of the CO₂ concentration sensed, is configured to control said CO₂ valve 254, by transmitting a control signal thereto, and thereby regulating the inflow of CO₂ gas in order to reach a desired and predetermined CO₂ concentration, and wherein subsequently, said gas mixing control system 270 on the basis of the O₂ concentration sensed, is configured to control said N₂ valve 252, by transmitting a control signal thereto, and thereby regulating the inflow of N₂ gas in order to reach a desired and predetermined O₂ concentration.

In an embodiment of the modular incubator system 500 according to the first aspect of the present invention, the gas mixing control system 270 is configured to use the input from said temperature sensor 262 for compensating the temperature sensitivity of said O₂ sensor 258.

In an embodiment of the modular incubator system 500 according to the first aspect of the present invention, the gas mixing control system 270 is configured to use the input from said pressure sensor 264 for compensating the pressure sensitivity of said CO₂ sensor 260.

In an embodiment of the modular incubator system 500 according to the first aspect of the present invention, the gas mixing control system 270 is being configured to maintain a pressure of gas supplied to said main gas supply line 210 of said gas distribution system 204, relative to the ambient atmospheric pressure, of 3-20 mbar, such as 5-18 mbar, such as 10-15 mbar above that ambient atmospheric pressure.

In an embodiment of the modular incubator system 500 according to the first aspect of the present invention, the gas mixing control system 270 is being configured to maintain a CO2 concentration of gas entering said main gas supply line 210 of said gas distribution system 204 in the range of 5-10%, such as 6-9% or 7-8%; and/or an O₂ concentration of gas entering said main gas supply line 210 of said gas distribution system 204 in the range of 5-10%, such as 6-9% or 7-8%.

In an embodiment of the modular incubator system 500 according to the first aspect of the present invention said modular incubator system 500 comprises a control unit 650 for controlling the operation of said modular incubator system 500.

In an embodiment said control unit 650 is being coupled to an input device 652, such as an alphanumerical input device for allowing a user to provide settings input relating to a desired operational protocol of said modular incubator system.

In an embodiment said control unit 650 is being coupled to a display unit 654 for displaying, to a user, information relating to settings and/or operational status of said modular incubator system 300.

In an embodiment of the modular incubator system 500 according to the first aspect of the present invention and in respect of one or more docking ports 402 of said docking station 400 said control unit 650 is being configured for independently controlling one or more of the following: the temperature in the interior 306 of said modular incubator chamber 300 by controlling said electric heating element 318, said thermostat 374 or said thermostatic circuit 376, provision of power to said electric power source 320; provision of signals to said display 324 of a modular incubator chamber 300 being docked in said docking port 402, switching on and off of said active light source 352 of a modular incubator chamber 300 being docked in said docking port 402, or adjusting light intensity emitted therefrom, said image capturing device 408 of a docking port 402, said displacement device 482 for displacing said image capturing device 408, said gas mixing control system 270; and said image processing unit 660.

When controlling a modular incubator chamber 300 this way, electric signals or electric power is accordingly being provides from the control unit 650 via the electric connectors 410 of the docking port 402 wherein the modular incubator chamber 300 is being docked and also via the electric connectors 322 of the modular incubator chamber 300.

In an embodiment said control unit 650 is being coupled to a data processing unit 656 and optionally also to a data storage 658 for aiding in handling information during controlling of said modular incubator system.

In an embodiment said control unit 650 is being configured for conducting automatic operation of said modular incubator system 500 by configuring said control unit 650 to independently control one or more of the following: the temperature in the interior 306 of said modular incubator chamber 300 by controlling said electric heating element 318, said thermostat 374 or said thermostatic circuit 376, provision of power to said electric power source 320; provision of signals to said display 324 of a modular incubator chamber 300 being docked in said docking port 402, switching on and off of said active light source 352 of a modular incubator chamber 300 being docked in said docking port 402, or adjusting light intensity emitted therefrom, said image capturing device 408 of a docking port 402, said displacement device 482 for displacing said image capturing device 408, said gas mixing control system 270; and said image processing unit 660.

The automatic operation may be conducted according to predetermined control criteria and instructions.

In an embodiment control unit 650 is being configured for enabling time lapse capturing of images by said image capturing devices 408.

In the above embodiments the operation of the modular docking system 500 can easily be controlled centrally.

### The Second Aspect of the Present Invention

In a second aspect the present invention relates to a modular incubator chamber 300 comprising:
a housing 302 having a first end 340 and a second end 342, thereby defining a longitudinal direction X between said first end and said second end;
wherein said housing comprises a lid 304, wherein said lid is being configured to be able to shift between an open configuration allowing access to the interior 306 of said modular incubator chamber 300 and a closed configuration, sealing off access to the interior of said modular incubator chamber;
wherein said modular incubator chamber 300, at said interior 306 thereof, comprises a culture dish support 308 for positioning a culture dish 310 with the view to accommodate one or more biological materials M within the housing 302 of said modular incubator chamber 300;
wherein said housing 302 of said modular incubator chamber 300 comprises a transparent window 316 for enabling capturing of images of a biological material M being accommodated in the interior thereof, through said transparent window;

In an embodiment of the modular incubator chamber 300 according to the second aspect of the present invention said incubator chamber 300 is comprising features as defined in respect of the modular incubator chamber 300 of the modular incubator system 500 according to the first aspect of the present invention.

### The Third Aspect of the Present Invention

In a third aspect the present invention relates to a docking station 400 for docking one or more modular incubator chambers 300; wherein said docking station comprises one or more docking ports 402 for receiving a housing 302 of one or more of said incubator chambers 300; wherein in respect of one or more docking ports 402 of said docking station, said docking port comprises an image capturing device 408 for capturing an image of the interior 306 of a modular incubator chamber 300, once being docked in said docking port 402.

In an embodiment of the docking station 400 according to the third aspect of the present invention said docking station 400 is comprising features as defined in respect of the docking station 400 of the modular incubator system 500 according to the first aspect of the present invention.

### The Fourth Aspect of the Present Invention

In a fourth aspect the present invention provides a use of a modular incubator system 500 according to the first aspect of the present invention for incubating a viable biological material.

In an embodiment of the use according to the fourth aspect of the present invention said biological material is being an oocyte or an embryo, such as a human oocyte or a human embryo.

### The Fifth Aspect of the Present Invention

In a fifth aspect the present invention provides a use of a modular incubator chamber 300 according to the second aspect of the present invention for incubating a viable biological material.

In an embodiment of the use according to the fifth aspect of the present invention said biological material is being an oocyte or an embryo, such as a human oocyte or a human embryo.

The Sixth Aspect of the Present Invention

In a sixth aspect the present invention provides a use of a docking station 400 according to according to the third aspect of the present invention for incubating a viable biological material.

In an embodiment of the use according to the sixth aspect of the present invention said biological material is being an oocyte or an embryo, such as a human oocyte or a human embryo.

### The Seventh Aspect of the Present Invention

In a seventh aspect the present invention provides method of incubating a viable biological material, wherein said method comprises:
i) providing a modular incubator system 500 according to the first aspect of the present invention;
ii) Providing a Viable Biological Material;
iii) arranging said viable biological material in a culture dish 310 and subsequently arranging said culture dish in the interior 306 of a modular incubator chamber 300 of said modular incubator station 400;
iv) docking said modular incubator chamber 300 in a docking port 402 of said docking station 400 of said incubator system 500;
v) allowing said viable biological material to be incubated in said modular incubator chamber 300;
vi) whenever desired, allowing said image capturing device 408 to capture one or more images of said biological material being accommodated in said culture dish 310.

In an embodiment of the method according to the seventh aspect of the present invention said method further comprising the step of:
viii) removing said incubator chamber 300 from said docking port 402 of said docking station 400, when desired, in order to manually inspect the viable biological material, and optionally also to remove, add or exchange growth medium/media in said culture dish 310.

It is noted that in the amended claims relating to the second aspect of the present invention, viz. the modular incubator chamber, reference is made that features of this modular incubator chamber may be as defined in respect of the claims relating to the first aspect of the present invention, viz. the modular incubator system.

This shall be construed to mean that embodiment of the modular incubator chamber per se may be as defined in the claims relating to embodiments of the modular incubator system.

This shall also be construed to mean that to the extent that an interrelationship between the modular incubator chamber and the docking station or a docking port thereof is defined in such embodiments relating to the modular incubator system, the corresponding embodiment of the modular incubator chamber, claimed by reference to the modular incubator system, shall be considered suitable to enter into such interrelationship.

Likewise, it is noted that in the amended claims relating to the third aspect of the present invention, viz. the docking station, reference is made that features of this docking station may be as defined in respect of the claims relating to the first aspect of the present invention, viz. the modular incubator system.

This shall be construed to mean that embodiment of the docking station per se may be as defined in the claims relating to embodiments of the modular incubator system.

This shall also be construed to mean that to the extent that an interrelationship between the modular incubator chamber and the docking station or a docking port thereof is defined in such embodiments relating to the modular incubator system, the corresponding embodiment of the docking station, claimed by reference to the modular incubator system, shall be considered suitable to enter into such interrelationship.

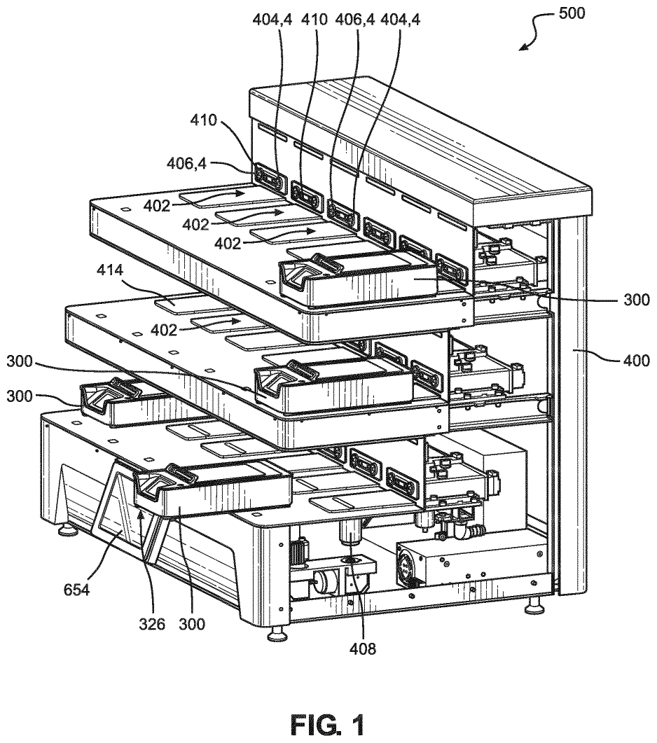

Referring now to the figures for better illustrating the present invention, FIG. 1 is a perspective view illustrating the general concept of designing an incubator as a modular incubator system comprising a plurality of modular incubator chambers in combination with a docking station having a plurality of docking ports.

Accordingly, FIG. 1 shows the modular incubator system 500 for incubating a viable biological material. The modular incubator system 500 comprising a docking station 400 in combination with a number of modular incubator chambers 300. The docking station 400 comprises a plurality of docking ports 402. Each docking port 402 is configured for receiving and holding a modular incubator chamber 300. Accordingly, each docking port 402 comprises docking port engagement means 414 which is configured to enter into engagement with a corresponding incubation chamber engagement means 326 arranged underneath each modular incubation chamber 300.

In FIG. 1 it is seen that the docking station 400 of the modular incubator system 500 comprises three shelves arranged above each other and each comprising six docking ports 402. Three of these docking ports 402 have been occupied by a modular incubator chamber 300 and a fourth modular incubation chamber 300 is on its way to be docked in a docking port 402.

FIG. 1 shows that the docking ports 402 of the docking station 400 is being provided with electric connectors 410. Hereby, electric power and/or electric signals can be conveyed between a docking port 402 of the docking station 400 and a modular incubator chamber 300 being docked therein.

Also seen in FIG. 1 is that the docking ports 402 comprise a docking port outlet opening for gas 404 comprising a docking port valve 4 and a docking port inlet opening for gas 406 comprising a docking port valve 4.

These openings for gas 404,406 and their associated valve 4 allow for conveying gas having a desired and predetermined and optimum gas composition through a modular incubator chamber 300 once this is being docked in a docking port 402 of the docking station 400 of the modular incubator system 500.

Hereby a predetermined and optimum gas composition can be maintained in the interior 306 of the modular incubator chambers 300.

The arrangement of an incubator for IVF procedures as an incubator system 500 comprising a plurality of modular incubator chambers 300 in combination with a docking station 400 allows for having, in a single apparatus, a relative huge number of incubations taking place under individual incubations environments, such as under an individual chemical environment in relation to e.g. gaseous atmosphere and composition of growth medium, and under an individual physical environment in relation to e.g. temperature.

Hereby it is possible to conduct a relatively large number of parallelly conducted incubations under similar conditions in the individual modular incubator chambers, while altering only one parameter from one modular incubator chamber to another. The difference in development of the viable biological material being incubated in the various modular incubator chambers, and hence the quality thereof, can then be assigned to the one incubation parameter that is altered from one modular chamber to the other.

This allows for determining optimum growth conditions of an embryo or an oocyte being incubated.

Whenever it is needed to change or add growth medium to the biological being incubated or whenever other manual procedures are needed in respect of a specific modular incubator chamber, that chamber 300 is simple removed from its respective docking port 402 of the docking station 400 and transferred to a laboratory bench where such manual procedures can be conducted.

As the embryo or oocyte is accommodated within the interior 306 of the modular incubator chamber when performing such manual procedures are conducted, only minor deviations from optimum incubation conditions are encountered by that embryo or oocyte during such manual procedures.

However, during most of the time, the modular incubator chamber 300 will be docked in a docking port 402 of the docking station 400.

Image capturing devices 408 are provided in respect of one or more of the docking ports 402 of the docking station 400. The image capturing device(s) 408 of the docking station 400 provide(s) for monitoring the morphological changes taking place during incubation.

The image capturing device(s) 408 may be configured for automatically capturing of images of the biological material being incubated in a modular incubator chamber 300.

In order to enable image capturing of a biological material being accommodated in the interior of a modular incubator chamber by an image capturing device being arranged in the docking station 400 it is clear that the modular incubator chamber must allow transmittal of light through the housing of the modular incubator chamber.

Such transmittal of light through the housing of the modular incubator chamber is attained by providing the housing of the modular incubator chamber 300 with a transparent window which allows that an image capturing device, located outside the interior of the chamber 300, may capture images of a viable biological material being accommodated in the interior of that incubator chamber 300. This is elaborated further below.

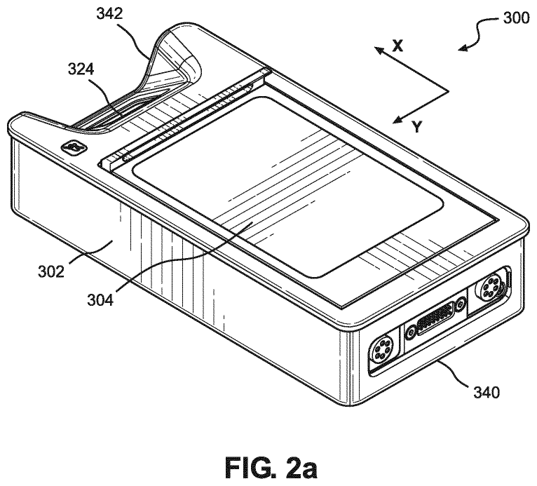

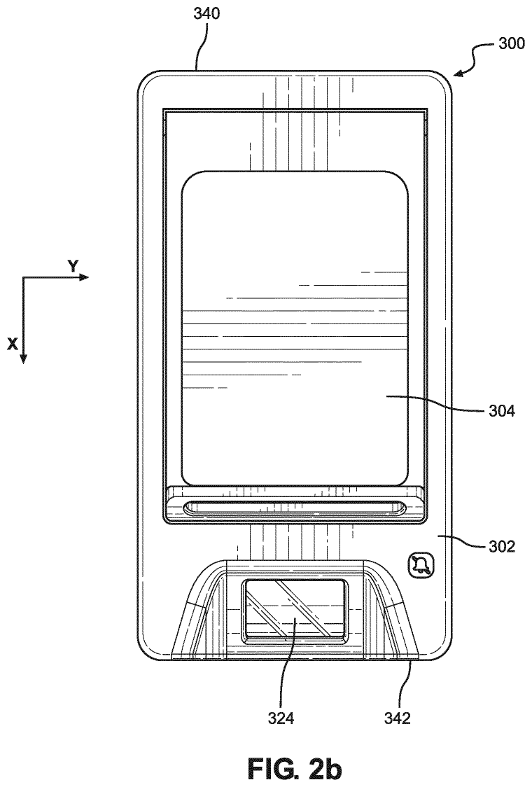

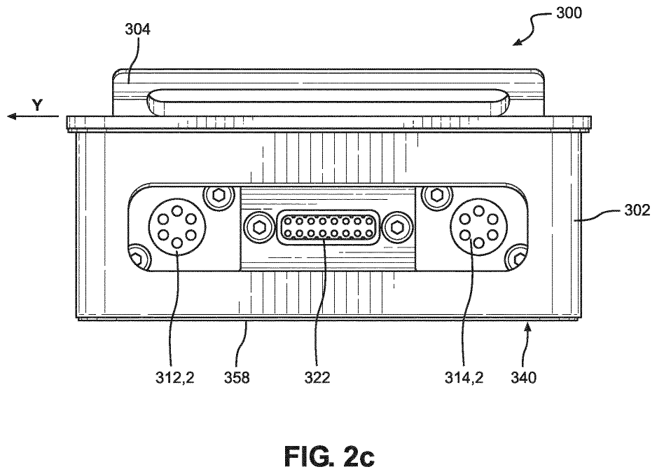

Details of the modular incubator chambers 300 of the docking system 500 according to the first aspect will now be described with reference to FIGS. 2a-2c, 3 and 4.

FIG. 2a is a top perspective view showing a modular incubator chamber of the modular incubator system of the invention. FIG. 2b is a top plan view showing the modular incubator chamber of FIG. 2a, and FIG. 2c is a rear plan view showing the modular incubator chamber of FIG. 2a and FIG. 2b.

FIGS. 2a, 2b and 2c illustrate the modular incubator chamber 300 comprising a housing 302.

The housing 302 is having a first end 340 and a second end 342 which define a longitudinal direction X between the first end and the second end. The housing comprises a lid which is being configured to be able to shift between an open configuration allowing access to the interior of the modular incubator chamber 300 and a closed configuration, sealing off access to the interior of said modular incubator chamber.

The lid 304 is a hinged lid which is being connected to the housing 302 of the modular incubator chamber via a hinge.

Also seen in FIGS. 2a and 2b is that the housing 302 of the modular incubator chamber 300 comprises a display 324 for displaying information relating to the incubation operation taken place in that incubator chamber 300.

FIG. 2c shows that the modular incubator chamber 300 at its first end 340 comprises an electric connector 322. This electric connector will enable conveying electric power or electric signals between a docking port 402 and modular incubator chamber 300 being docked therein via corresponding electric connector 410 of the docking port 402.

FIG. 2c also shows that the modular incubator chamber 300 comprises a modular incubator chamber inlet opening for gas 312 with a incubator chamber valve 2, and a modular incubator chamber outlet opening for gas 314 with a incubator chamber valve 2. Hereby gas having a predetermined and preferred and optimum gas composition can be directed from a docking port 402 and into the interior 306 of a modular incubator chamber 300, and from the interior 306 of a modular incubator chamber 300 to the docking port 402. This is elaborated in further detail below.

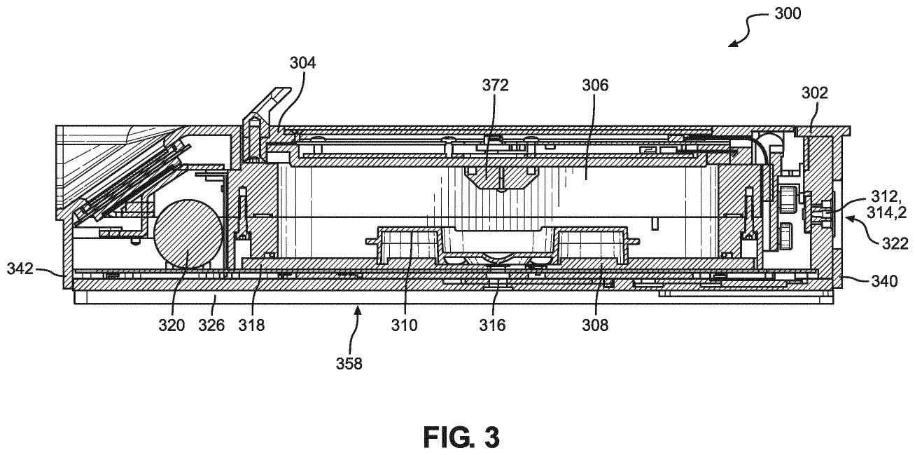

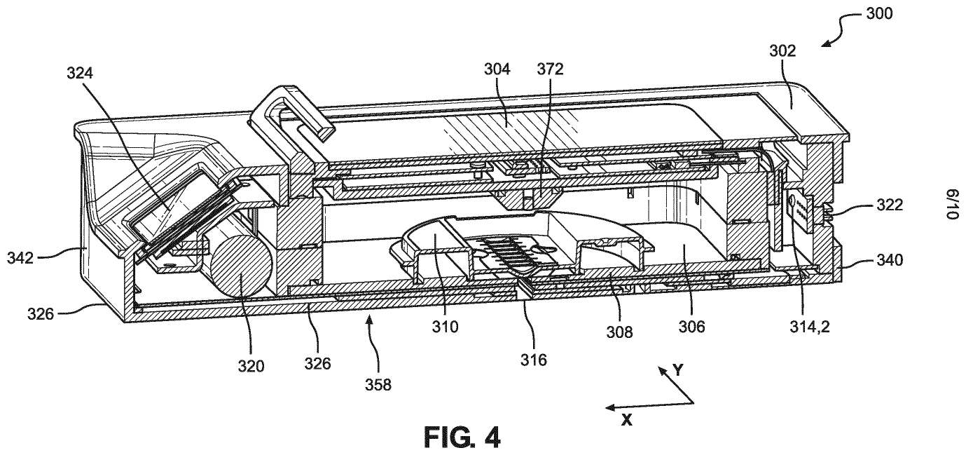

FIGS. 3 and 4 are a cross-sectional plan view and perspective view, respectively, illustrating the the modular incubator chamber illustrated in FIGS. 2a, 2b and 2c.

FIGS. 3 and 4 show the modular incubator chamber with the lid 304 and a culture dish support 308 arranged at the interior 306 of the housing 302 of the modular incubator chamber 300.

The culture dish support 308 allows for positioning a culture dish 310 in the interior 306 of the modular incubator chamber 300 with the view to accommodate one or more biological materials M within the housing 302 of that modular incubator chamber 300.

As already suggested and with reference to FIG. 1, the docking station 400 of the modular incubator system 500 of the first aspect of the present invention comprises one or more docking ports 402 for receiving a housing 302 of one or more of the incubator chambers 300. In respect of one or more docking ports 402 of the docking station 400, the docking port comprises an image capturing device 408 for capturing an image of the interior 306 of a modular incubator chamber 300, once that modular incubator chamber is being docked in that docking port 402.

FIGS. 3 and 4 show that the housing 302 of the modular incubator chamber 300 comprises a transparent window 316 for allowing capturing of images of a biological material being accommodated in the interior thereof, through said transparent window. As seen the window is arranged at the bottom 358 of the housing 302 of the modular incubator chamber 300.

FIG. 4 shows that the housing 302 of the modular incubator chamber 300 comprises a transparent window 316 in the form of an elongate and linear opening extending in a direction Y which is perpendicular to the longitudinal direction X of the housing of the modular incubation chamber 300.

Designing the transparent window 316 of the housing 302 as an elongate shape allows for capturing images of a plurality of viable biological materials being accommodated in line in a culture dish 310 which is being accommodated in the housing 302 of a modular incubator chamber 300 as shown in FIG. 4.

The modular incubator chamber 300 also comprises, in its interior 306, an electric heating element 318 for heating the interior of said modular incubator chamber. The modular incubator chamber also comprises a power source 320 in the form of a rechargeable battery for providing power to said heating element 318 which is being electrically connected to the power source 320. A light source 372 is attached to an inner side of the lid 304 of the modular incubator chamber 300 for providing illumination of the viable biological material being incubated in the modular incubator chamber 300 during image capturing.

The culture dish support 308 is for positioning a culture dish 310. Hereby, one or more biological materials can be accommodated and incubated within the housing 302 of the modular incubator chamber 300.

Also seen in FIGS. 3 and 4 is the chamber engagement means 326 which is being adapted to engage with docking port engagement means 414 of the docking port 402 into which the modular incubator chamber 300 is to be docked.

When such a proper positioning of the of the modular incubator chamber 300 in the docking port 402 has been attained via the chamber engagement means 326 of the chamber 300 and via the docking port engagement means 414 of the docking port 402, the relative position of the two electric connectors 410 and 322 of the docking port 402 and the modular incubator chamber 300, respectively, will match pairwise so as to allow electric connection between the connectors 410 and 322.

Accordingly, with the modular incubator system 500 of the present invention, viable biological materials can be incubated in one or more modular incubators 300 which are being docked in docking ports 402 of the docking station 400, and at the same time visual monitoring of the biological material can be conducted via the image capturing device 408.

Capturing of images while the modular incubators 300 are being docked in a docking port minimized any physical impacts and other disturbances which would otherwise encountered by a viable biological material being incubated in a situation where the culture dish accommodating the biological material would have to be moved from its incubator to a laboratory bench for manually performing microscopic image capturing with the view to assess the morphological development of the biological material.

Also, when the viable biological material in its culture dish 310 will have to be moved to a laboratory bench for performing various manipulation steps, this can be done while the biological material in its culture dish 310 is accommodated in its associated modular incubator chamber 300, thereby minimizing deviations from the optimum incubation conditions, such as the environment, which is being present in that modular incubator chamber 300.

The present invention thereby allows for incubation of a biological material in the modular incubator chamber 300 and at the same time allows for visual monitoring of the morphological development of the biological material, with a minimum of physical impact imposed on the biological material.

It should be noted that in respect of a number N of adjacently arranged docking ports 402 of the docking station 400, these adjacently arranged docking ports 402 may share a common image capturing device 408 in the sense that one and only one image capturing device is responsible for capturing images relating to a modular incubator chamber 300 which is being docked in one of these N adjacently arranged docking ports 402.

In such a situation, a displacement device 482 in the form of a motorized suspension of the image capturing device 408 is being configured to be displaced, upon receiving a signal thereto, along a displacement track extending below these N number of adjacent docking ports 402 for enabling displacement of that common image capturing device 408 in relation to the N adjacently arranged docking ports 402 of the docking station 400. Thereby the common image capturing device 408 will be able to capture images of a biological material being accommodated in the interior 306 of a modular incubator chamber 300 being docked in any of said N docking ports 402 of the docking station 400.

When a proper positioning of the of the modular incubator chamber 300 in the docking port 402 has been attained, the relative position of the two inlet openings 312 and 406 with their respective valves 2,4 and the two outlet openings 314,404 with their respective valves 2,4 will match pairwise so that passage of gas from the docking port outlet opening for gas 404 into the interior 306 of the modular incubator chamber 300 via the modular incubator chamber inlet opening for gas 312 is enabled, and so that passage of gas from the interior 306 of the modular incubator chamber 300 is possible via the modular incubator chamber outlet opening for gas 314 and the the docking port inlet opening for gas 406.

Accordingly, the modular docking system 500 of the present invention allows for continuously providing gas into the interior 306 of the modular incubator chamber from a gas source 202.

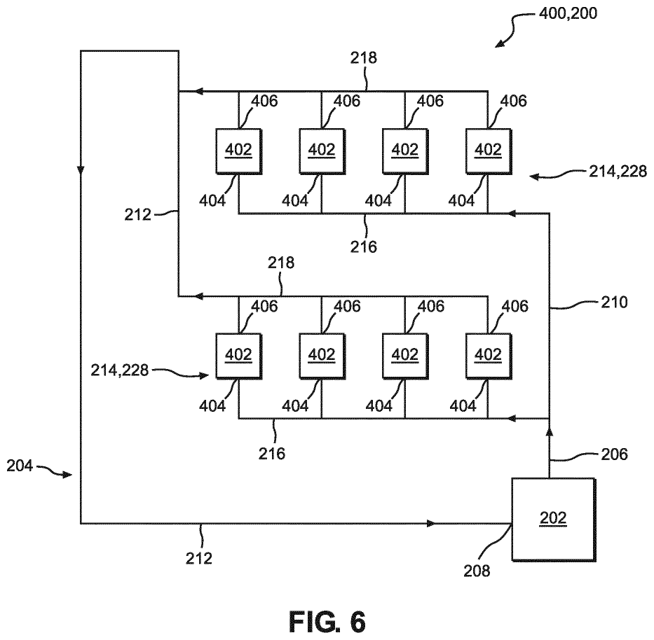

### This is further illustrated with reference to FIGS. 6, 7 and 8.

However, first we turn to a more detailed description of a valve system to be used with the modular incubator system according to the first aspect of the present invention.

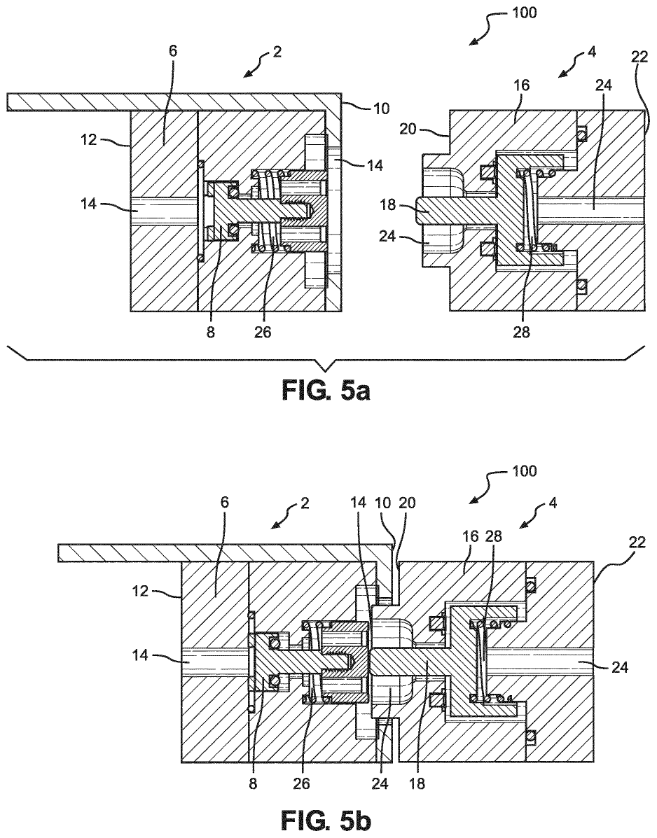

FIGS. 5a and 5b illustrate the working modes of valves of a valve system to be used with the modular incubator chamber and the associated docking port of the docking station of the docking system of the present invention.

FIG. 5a is diagrammatic drawing illustrating a valve system 100 to be used with the modular incubator system of the present invention, where the two valves 2,4 of the valve system 100 are not engaged with each other, thereby attaining a closed configuration.

FIG. 5b is diagrammatic drawing illustrating the valve system 100 seen in FIG. 5a in which the two valves 2,4 of the valve system 100 are engaged with each other, thereby attaining an open configuration.

The valve 2 comprises a valve body 6 having a front end 10 and a rear end 12. A through-going channel 14 is arranged in the valve body 6 and a valve element 8 is being arranged in the through-going channel 14. The valve element 6 is spring-loaded by a spring 26.

The displaceable valve element 8 is being configured to be displaced in the through-going channel 14 of the valve body 6 by the spring 26 in such a way, that when not acted upon by an external force, the spring-loaded displaceable valve element 8 is being displaced in the through-going channel 14 of said valve body 6 by the spring 26 towards the front end 10 of the valve body 6. Thereby the valve 2 attains a closed configuration blocking passage of gas through the through-going channel 14.

This situation is illustrated in FIG. 5a.

By analogy, when acted upon by an external force, the spring-loaded displaceable valve element 8 is being displaced in the through-going channel 14 of said valve body 6 towards the rear end 12 of the valve body 6, thereby making the valve 2 attain an open configuration, allowing passage of gas through said through-going channel 14.

This situation is illustrated in FIG. 5b.

As to the valve 4, FIG. 5a shows that the valve 4 comprises a valve body 16 having a front end 20 and a rear end 22. A through-going channel 24 is arranged in the valve body 16 and a valve element 18 is being arranged in the through-going channel 24. The valve element 18 is spring-loaded by a spring 28.

The displaceable valve element 18 is being configured to be displaced in the through-going channel 24 of the valve body 16 by the spring 28 in such a way, that when not acted upon by an external force, the spring-loaded displaceable valve element 18 is being displaced in the through-going channel 24 of said valve body 16 by the spring 28 towards the first end 20 of the valve body 16. Thereby the valve 4 attains a closed configuration blocking passage of gas through the through-going channel 24.

This situation is illustrated in FIG. 5a.

By analogy, when acted upon by an external force, the spring-loaded displaceable valve element 18 is being displaced in the through-going channel 24 of said valve body 16 towards the rear end 22 of the valve body 16, thereby making the valve 4 attain an open configuration, allowing passage of gas through said through-going channel 24.

This situation is illustrated in FIG. 5b.

The valves 2 of the modular incubator chamber 300 and the valves 4 of the docking port 402 of the docking station are having dimensions and geometries in such a way that once docking the modular incubator chamber 300 in the docking port 402 of said docking station 400, the displaceable valve element 8 of the valve 2 and the displaceable valve element 18 of the valve 4 will displace each other into their respective valve bodies 6,16, thereby opening the valves 2,4 of said docking port outlet opening for gas 404 and said chamber inlet opening for gas 312; and also opening the valves 2,4 of the chamber outlet opening for gas 314 and the docking port inlet opening for gas 406, as seen in FIG. 5a.

Accordingly, using such valves 2,4 for the modular incubator chamber 300 and for the docking ports 402 of the docking station will automatically provide for opening the valves 2 of the modular incubator chamber 300 and the valves 4 of docking port 402, once that modular incubator chamber 300 is being docked in that docking port 402, thereby allowing passage of gas through the interior 306 of the modular incubator chamber 300, when being docking in the docking port 402 and also shutting off supply of gas into the docking port 402 and out of the modular incubator chamber 300, when that modular incubator chamber 300 is removed from the docking port.

It should be noted, that whereas the present description and the appended claims describe the modular incubator system 300 and the docking ports 402 in a way where the valves 2 are being arranged in the modular incubator system 300 and in a way where the valves 4 are being arranged in the docking ports 402, the opposite placement of the valves 2,4 is also possible.

In the above sections the general principle of a modular incubator system 500 comprising a docking station 400 with a plurality of docking ports 402 for receiving, by docking, a modular incubator chamber 300 has been described. In the sections below focus will be directed to features of supplying gas to the docking ports 402 of the docking station 400.

FIG. 6 is a diagram illustrating the concept of the gas supply system which may be incorporated in the docking station of the modular incubator system of the present invention. FIG. 6 shows a gas supply system 200 to be used with a modular incubator system 500 according to the present invention. The gas supply system 200 comprises a gas source 202 and a gas distribution system 204.

The gas distribution system 204 comprises a plurality of docking ports 402 each having a docking port outlet opening for gas 404 and a docking port inlet opening for gas 406.

In respect of all the docking ports, the docking port outlet openings for gas 404 are in fluid connection with an inlet manifold 216 and the docking port inlet openings for gas 406 are in fluid connection with an outlet manifold 218.

A main gas supply line 210 supplies gas from a supply gas outlet 206 of the gas source 202 to the inlet manifolds 216, and a main gas return line 212 returns gas from the outlet manifolds 218 to a return gas inlet 208 of the gas source 202.

Thereby gas can be circulated from the gas source 202 via the gas distribution system 204 to the docking ports 402 and back to the gas source 202.

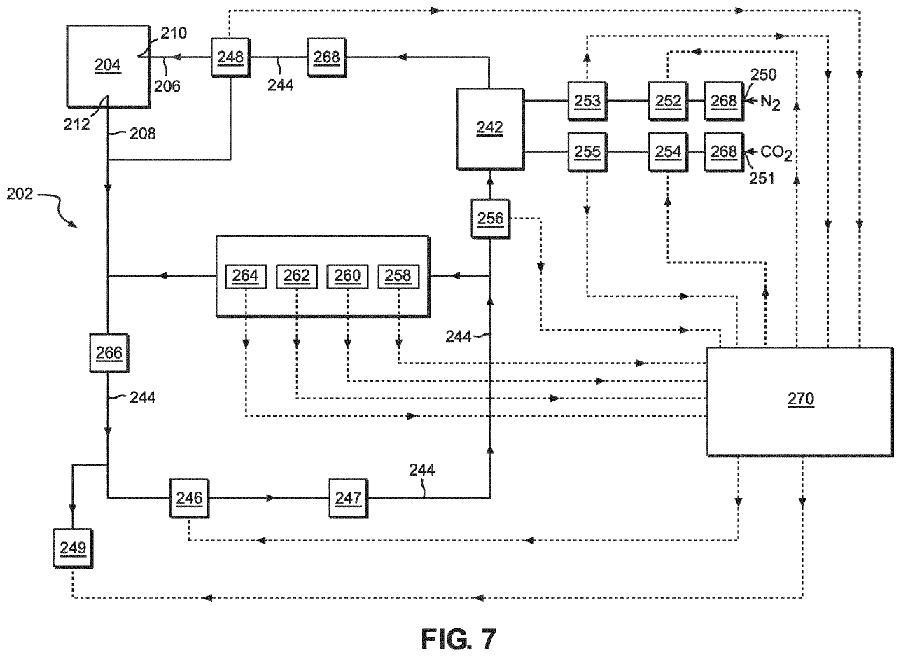

In order to secure a desired and predetermined and optimum gas composition of the gas supplied to the docking stations, the gas source is provided with specific features as disclosed with reference to FIG. 7.

FIG. 7 is a diagram illustrating one embodiment of a design of a gas supply system comprising a gas source to be used with the docking station of the modular incubator system of the present invention.

In FIG. 7 solid lines represent flow lines for gas, whereas dashed lines represent signal lines for conveying electric signals or electric power.

FIG. 7 shows the gas distribution system 204 comprising its main gas supply line 210 and its main gas return line 212 (illustrated with the rectangle in upper left corner).

The main gas supply line 210 and the main gas return line 212 of the gas distribution system 204 is fluidly connected to a gas source 202 as described below.

The gas source 202 of said gas supply system 200 comprises a gas mixing box 242 connected to the supply gas outlet 206 and the return gas inlet 208 of the gas source.

The main gas supply line 210 of the gas distribution system 204 is being fluidly connected to the supply gas outlet 206, and the main gas return line 212 of the gas distribution system 204 is being fluidly connected to the return gas inlet 208 of the gas source 202.

Hereby a flow loop 244 comprising the gas distribution system 204 and the gas mixing box 242 is formed. The flow loop 244 comprises a pump 246 for circulating gas in that loop.

It is seen that the pump 246 is being arranged downstream in relation to the main gas return line 212. Also seen in FIG. 7 is that the flow loop 244 comprises a pump oscillation damper 247, which is being arranged immediately downstream in relation to the pump 246.

Moreover, the flow loop 244 comprises a pressure sensor 248 in the form of a differential pressure sensor for sensing the pressure of gas supplied to the main gas supply line 210, relative to the pressure in the return gas inlet line 208 of the gas distribution system 204. The pressure senor 248 is being arranged immediately upstream in relation to the main gas supply line 210 of the gas distribution system 204.

The flow loop 244 further comprises a release valve 249 for enabling pressure relief in the flow loop. The release valve is arranged immediately downstream in relation to the main gas return line 212 of the gas distribution system 402.

Also seen in FIG. 7 is that the gas mixing box 242 comprises an inlet for N₂ gas 250; and an inlet for CO₂ gas 251.

The inlet for N₂ gas 250 is fluidly connected to an N₂ valve 252 for regulating the inflow of N₂, and an N₂ mass flow sensor 253 arranged downstream of the N₂ valve 252 for sensing the amount of N₂ flowing into said gas mixing box 242.

The inlet for CO₂ gas 251 is fluidly connected to a CO₂ valve 254 for regulating the inflow of CO₂, and an CO₂ mass flow sensor 255 arranged downstream of the CO₂ valve 254 for sensing the amount of CO₂ flowing into the gas mixing box 242.

The flow loop 244 also comprises a mass flow sensor 256 arranged at an upstream position in relation to the gas mixing box 242 for sensing the amount of return gas entering the gas mixing box.

It is seen that the gas source 202 comprises an O₂ sensor 258 for sensing the concentration of O₂ exiting the gas distribution system 204; and that the gas source 202 comprises a CO₂ sensor 260 for sensing the concentration of CO₂ exiting the gas distribution system 204.

The O₂ sensor and the CO₂ sensor is arranged downstream in relation to the pump 246.

A temperature sensor 262 for sensing the temperature of gas circulating in said flow loop 244 is included in the gas source 202. The temperature sensor is arranged downstream in relation to the pump 246 at a position corresponding to the position of the O₂ sensor 258.

A pressure sensor 264 for sensing the absolute pressure in the flow loop 244 is included in the gas source 202. This pressure sensor is arranged downstream in relation to the pump 246, at a position corresponding to the position of the CO₂ sensor 260.

Also seen in FIG. 7 is that the flow loop 244 comprises a UV sanitizer 266 for sanitizing gas flowing in the flow loop 244 via electromagnetic radiation in the UV range. The UV sanitizer is arranged immediately downstream in relation to the main gas return line 212.

It is seen in FIG. 7 that the gas source 202 comprises filters 268 in the form of HEPA/VOCs filters. One such a filter is arranged immediately upstream in relation to the main gas supply line 210. Another such a filter is arranged immediately upstream in relation to the inlet for N₂ gas 250 into the gas mixing box 242; and a third such a filter is being arranged immediately upstream in relation to the inlet for CO₂ gas 251 into the gas mixing box 242.

Finally, it is seen in FIG. 7 that the gas source 202 comprises a gas mixing control system 270.

It is seen that the gas mixing control system 270 is electrically connected to one or more of the following sensors for receiving sensing signals therefrom: the N₂ mass flow sensor 253 for sensing the amount of N₂ flowing into the gas mixing box; the CO₂ mass flow sensor 255 for sensing the amount of CO₂ flowing into the gas mixing box; the mass flow sensor 256 for sensing the amount of return gas entering the gas mixing box; the O₂ sensor 258 for sensing the concentration of O₂ exiting the main gas return line 212 of the gas distribution system 204; the CO₂ sensor 260 for sensing the concentration of CO₂ exiting the main gas return line 212 of the gas distribution system 204; the temperature sensor 262 for sensing the temperature circulating in the flow loop 244; the pressure sensor 264 for sensing an absolute pressure in the flow loop 244, the pressure sensor 248 for sensing the pressure of gas supplied to the gas main gas supply line 210 of the distribution system 204.

Also seen in FIG. 7 is that the gas mixing control system 270 is electrically connected to one or more of the following elements for control thereof: the N₂ valve 252 for regulating the inflow of N₂ into the gas mixing box 242; the CO₂ valve 254 for regulating the inflow of CO₂ to the gas mixing box 242; the pump 246 for circulating gas in the flow loop 244; the release valve 249.

The control of the gas source by the gas mixing control system 270 is performed in accordance with two control regimes. The first control regime relates to controlling the pressure of gas exiting the supply gas outlet 206, and the second control regime relates to controlling the concentration of CO₂ and O2 of gas exiting the supply gas outlet 206. The two control regimes are conducted concurrently. This is further explained below.

The gas mixing control system 270 is being configured to receive input from the pressure sensor 248 and on the basis thereof control the pump 246 and optionally also activate the release valve 249 in order to maintain a desired and predetermined pressure of gas supplied to the main gas supply line 210 of the gas distribution system 204.

The gas mixing control system 270 is further configured to receive input from the mass flow sensor 256, and on the basis of this input to determine the total amount of CO₂ gas and N₂ gas needed to be supplied via the inlet for CO₂ gas 251 and via the inlet for N₂ gas 250 according to desired and predetermined criteria.

Based on the information relating to the total amount of CO₂ gas and N₂ gas needed to be supplied, as described above, the gas mixing control system 270 will be able to determine the mutual proportion of the CO₂ gas and N₂ gas to be supplied to the gas mixing box 242.

This is performed by receiving input from the CO₂ sensor 260 and the O₂ sensor 258.

On the basis of the CO₂ concentration sensed, the gas mixing control system 270 will control the CO₂ valve 254, by transmitting a control signal thereto, and thereby regulate the inflow of CO₂ gas in order to reach a desired and predetermined CO₂ concentration.

Subsequently, the gas mixing control system 270 will on the basis of the O₂ concentration sensed, control the N₂ valve 252, by transmitting a control signal thereto, and thereby regulate the inflow of N₂ gas in order to reach a desired and predetermined O₂ concentration.

By using the gas source as disclosed above, a constant circulation of gas will be supplied to one or more modular incubator chamber 300 being docked in a respective docking port 402 of the docking station 400. By constantly regulating the inflow of CO₂ gas and N₂ gas based on sensed concentration of CO₂ and O₂ in the return gas from the gas distribution system 204, an optimum and predetermined gas composition can be maintained.

Due the design of the gas distribution system 204, an constant composition of gas flowing through each modular incubator chamber 300 can be upheld.

It should be noted that when reference is made to an upstream position relative to another position, that upstream position is construed to mean a position still within the gas source 202 and preferably not so much upstream that it passes the gas mixing box 242 or the gas distribution system 204.

Likewise, when reference is made to a downstream position relative to another position, that downstream position is construed to mean a position still within the gas source 202 and preferably not so much downstream that it passes the gas mixing box 242 or the gas distribution system 204.

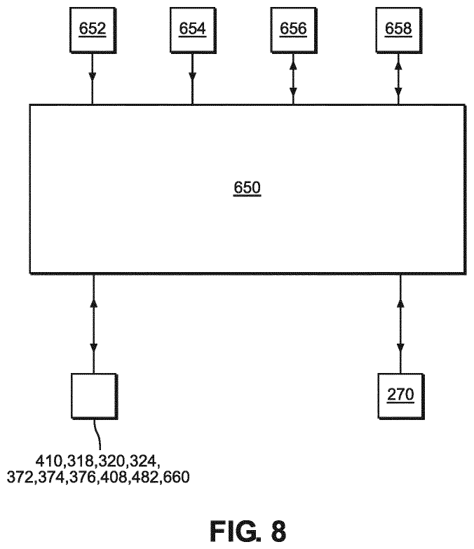

The modular incubator system 500 may comprise a control unit for controlling thereof. This is further illustrated with reference to FIG. 8.

FIG. 8 is a diagram illustrating the working mode of the controlling of the modular incubator system according to the invention.

FIG. 8 shows the control unit 650 for controlling the operation of the modular incubator system 500. The control unit is coupled to an input device 652 in the form of an alphanumerical input device for allowing a user to provide settings input relating to a desired operational protocol of said modular incubator system.

A display unit 654 for displaying, to a user, information relating to settings and/or operational status of one or more of the modular incubator chambers 300 is coupled to the control unit 654.

It is seen that the control unit 650 is coupled to a couple of electric connectors 410 of the docking ports 402 of the docking station 400. Thereby electrical power and electric signals can be provided to one or more modular incubator chambers 300 which is/are being docked into a docking port 402 of the docking station 400 of the modular incubator system 500.

By being connected to the docking ports 402 of the docking station 400 it will be possible, when one or more modular incubator chambers 300 is/are docked into a docking port 402 of the docking station 400, and by using the control unit 650, to control the following entities or parameters: the temperature in the interior 306 of said modular incubator chamber 300 by controlling said electric heating element 318, said thermostat 374 and/or said thermostatic circuit 376, provision of power to said electric power source 320; provision of signals to said display 324 of a modular incubator chamber 300 being docked in said docking port 402, switching on and off of said active light source 352 of a modular incubator chamber 300 being docked in said docking port 402, or adjusting light intensity emitted therefrom, controlling said image capturing device 408 of a docking port 402, and optionally said associated displacement device 482 for displacing said image capturing device 408, said gas mixing control system 270; and said image processing unit 660.

The control unit 650 may comprises or may be coupled to a CPU or other data processor 656 for processing the information involved in controlling the operation of the modular incubator system 500, e.g. by involving a computer program for handing the information involved in the controlling of the operation and the control unit 650 may also comprise or may be coupled to a data storage 658.

Thereby automatic operation of the modular incubator system 500 may be performed in the sense that control unit 650 may independently and automatically, according to predetermined criteria, control one or more of the following: the temperature in the interior 306 of said modular incubator chamber 300 by controlling said electric heating element 318, said thermostat 374 and/or said thermostatic circuit 376, provision of power to said electric power source 320; provision of signals to said display 324 of a modular incubator chamber 300 being docked in said docking port 402, switching on and off of said active light source 352 of a modular incubator chamber 300 being docked in said docking port 402, or adjusting light intensity emitted therefrom, controlling said image capturing device 408 of a docking port 402 and optionally said associated displacement device 482 for displacing said image capturing device 408, said gas mixing control system 270; and said image processing unit 660.

It should be understood that all features and achievements discussed above and in the appended claims and clauses in relation to one aspect of the present invention and embodiments thereof apply equally well to the other aspects of the present invention and embodiments thereof.

The present invention may be defined according to one or more of the following clauses:

Clause 1. A modular incubator system (500) for incubating a viable biological material M, said modular incubator system comprising:
one or more modular incubator chambers (300) in combination with
a docking station (400);
   wherein in respect of one or more of said one or more modular incubator chambers (300), said modular incubator chamber (300) comprises a housing (302) having a first end (340) and a second end (342), thereby defining a longitudinal direction X between said first end and said second end;
   wherein said housing comprises a lid (304), wherein said lid is being configured to be able to shift between an open configuration allowing access to the interior (306) of said modular incubator chamber and a closed configuration, sealing off access to the interior of said modular incubator chamber;
   wherein said modular incubator chamber (300), at said interior (306) thereof, comprises a culture dish support (308) for positioning a culture dish (310) with the view to accommodate one or more biological materials M within the housing (302) of said modular incubator chamber (300);
   wherein said housing (302) of said modular incubator chamber (300) comprises a transparent window (316) for enabling capturing of images of a biological material M being accommodated in the interior thereof, through said transparent window;
   wherein said docking station (400) comprises one or more docking ports (402) for receiving a housing (302) of one or more of said incubator chambers (300);
   wherein in respect of one or more docking ports (402) of said docking station (400), said docking port comprises an image capturing device (408) for capturing an image of the interior (306) of a modular incubator chamber (300), once being docked in said docking port (402).

Clause 2. A modular incubator system (500) according to clause 1, wherein in respect of one or more of said one or more modular incubator chambers (300) and in respect of one or more of said one or more docking ports (402) of said docking station (400), the position of said transparent window (316) of said modular incubator chamber (300) is adapted to the position of said image capturing device (408) in said docking port (402) in a way that enables capturing of images by said image capturing device (408) through said transparent window (316) of said modular incubator chamber (300), once said modular incubator chamber (300) is being docked in said docking port (402).

Clause 3. A modular incubator system (500) according to clause 1 or 2, wherein in respect of one or more of said one or more modular incubator chambers (300), said transparent window (316) of said modular incubator chamber (300) is arranged at a bottom part (358) of said housing (302).

Clause 4. A modular incubator system (500) according to any of the preceding clauses, wherein in respect of one or more of said one or more modular incubator chambers (300), said culture dish support (308) of said modular incubator chamber (300) is being arranged above said transparent window (316).

Clause 5. A modular incubator system (500) according to any of the preceding clauses, wherein in respect of one or more of said one or more modular incubator chambers (300), said transparent window (316) of said housing (302) of said modular incubator chamber is having an elongate shape, such as an elongate and linear extension extending in a direction Y, transversal to said longitudinal direction X of said housing of said modular incubation chamber (300).

Clause 6. A modular incubator system (500) according to any of the preceding clauses, wherein in respect of one or more of said one or more modular incubator chambers (300) and in respect of one or more of said one or more docking ports (402) of said docking station (400), said modular incubator chamber (300) is being configured to be docked in said docking port (402) with its first end (340) facing said docking port (402).

Clause 7. A modular incubator system (500) according to any of the preceding clauses, wherein in respect of one or more of said one or more modular incubator chambers (300), said modular incubator chamber (300), in the interior (306) thereof, comprises a light source (372) for directing light to the area of the culture dish support (308) of said modular incubator chamber (300), thereby enabling illumination of a viable biological material in a situation of capturing images of said viable biological material.

Clause 8. A modular incubator system (500) according to clause 7, wherein said light source (372) is being attached to said lid (304) of the housing (302) of said modular incubator chamber (300), at an inner side thereof.

Clause 9. A modular incubator system (500) according to clause 7 or 8, wherein said light source (372) is being selected from the group of one or more LEDs, one or more laser diodes, one or more incandescent light bulbs.

Clause 10. A modular incubator system (500) according to any of the preceding clauses, wherein in respect of one or more of said one or more modular incubator chambers (300), said culture dish support (308) is defining a planar support surface for supporting said culture dish (310).

Clause 11. A modular incubator system (500) according to any of the preceding clauses, wherein in respect of one or more of said one or more modular incubator chambers (300), said housing (302) of said modular incubator chamber (300), such as at an outer portion thereof, is being provided with electric connectors (322) for providing electric power and/or electric signals to said modular incubator chamber; and wherein in respect of one or more docking ports (402) of said docking station (400), said docking port is being provided with electric connectors (410), thereby allowing providing electric power and/or electric signals between said docking port (402) of said docking station (400) and a modular incubator chamber (300) being docked therein.

Clause 12. A modular incubator system (500) according to any of the preceding clauses, wherein in respect of one or more of said one or more modular incubator chambers (300), said lid (304) is being a hinged lid which is being connected to said housing of said modular incubator chamber via a hinge.

Clause 13. A modular incubator system (500) according to any of the preceding clauses, wherein in respect of one or more of said one or more modular incubator chambers (300), said housing (302) of said modular incubator chamber (300) comprises a display (324) which is being configured to display information relating to an operational status of the incubation taking place in said modular incubator chamber.

Clause 14. A modular incubator system (500) according to any of the preceding clauses, wherein said image capturing device (408) comprises microscopic optics so as to enable capturing of microscope images.

Clause 15. A modular incubator system (500) according to any of the preceding clauses, wherein the number of modular incubator chambers (300) of said modular incubator system (500) is selected from the ranges 1-100, such as 2-95, for example 5-90, e.g. 10-85, such as 15-80, for example 20-75, e.g. 25-70, 30-65, such as 35-60, e.g. 40-55 or 45-50.

Clause 16. A modular incubator system (500) according to any of the preceding clauses, wherein the number of docking ports (402) in said docking station (400) of said modular incubator system (500) is selected from the ranges 1-100, such as 2-95, for example 5-90, e.g. 10-85, such as 15-80, for example 20-75, e.g. 25-70, 30-65, such as 35-60, e.g. 40-55 or 45-50.

Clause 17. A modular incubator system (500) according to any of the preceding clauses, wherein said docking station (400) comprises said docking ports (402) in an arrangement of one or more shelves of adjacently positioned docking ports (402), wherein in case said docking station comprises two or more shelves, said shelves are being arranged above each other.

Clause 18. A modular incubator system (500) according to any of the preceding clauses, wherein in respect of one or more of said one or more modular incubator chambers (300), said modular incubator chamber comprises an incubation chamber engagement means (326) and wherein in respect of one or more docking ports (402) of said docking station (400), said docking port comprises a docking port engagement means (414), wherein said incubation chamber engagement means (326) is being configured to enter into engagement with said docking port engagement means (414) so as to provide ease and proper positioning and optionally also fixing said modular incubator chamber (300) in said docking port (402), as well as detaching said modular incubator chamber (300) from said docking port (402) of said docking station (400).

Clause 19. A modular incubator system (500) according to any of the preceding clauses, wherein said modular incubator system (500) comprises an image processing unit (660) for image processing of images captured by said image capturing device (408), wherein said modular incubator system (400) optionally furthermore comprises a data storage (658) for storing images captured by said image capturing units (408) and/or for storing images processed by said image processing unit (660).

Clause 20. A modular incubator system (500) according to clause 19, wherein one or more of said image capturing devices (408) of said docking ports (402) of said docking station is/are being coupled to an image processing unit (660).

Clause 21. A modular incubator system (500) according to any of the preceding clauses, wherein in respect of one or more specific docking ports (402) of said docking station (400), said specific docking port comprises its own dedicated image capturing device (408) which is configured to only capture images relating to a modular incubator chamber (300) which is being docked in said specific docking port (402).

Clause 22. A modular incubator system (500) according to any of the preceding clauses, wherein in respect of a number N of adjacently arranged docking ports (402) of said docking station (400), said adjacently arranged docking ports share a common image capturing device (408) in the sense that one and only one image capturing device is responsible for capturing images relating to a modular incubator chamber (300) which is being docked in one of said N adjacently arranged docking ports (402), wherein said docking station comprises a displacement device (482) for enabling displacement of said common image capturing device (408) in relation to said N adjacently arranged docking ports (402) of said docking station (400).

Clause 23. A modular incubator system (500) according to clause 22, wherein said number N is being an integer selected in the ranges of 2-25 or more, such as 4-22, for example 6-20, such as 8-18, such as 10-16 or 12-14.

Clause 24. A modular incubator system (500) according to any of the preceding clauses, wherein in respect of one or more of said modular incubator chambers (300), said modular incubator chamber comprises in its interior (306) an electric heating element (318) for heating the interior of said modular incubator chamber, and wherein said modular incubator chamber comprises a power source (320) for providing power to said heating element (318), wherein said electric heating element (318) is being electrically connected to said power source (320). Clause 25. A modular incubator system (500) according to clause 24, wherein said power source (320) is being an electric power source, such as a battery, for example a rechargeable battery.

Clause 26. A modular incubator system 500 according to any of the clauses 24 or 25, wherein said heating element 318 is being thermally connected to a heat distribution element for distributing heat dissipated in said heating element; wherein said heat distribution element is being arranged, at least partly, in the interior 306 of said modular incubator chamber 300.

Clause 27. A modular incubator system (500) according to any of the clauses 24-26, wherein said chamber comprises a thermostat (374) and an electric thermostatic circuit (376), wherein said electric heating element (318), said power source (320) and said thermostat (374) are being electrically connected in said electric thermostatic circuit (376) so as to enable thermostatic control of the temperature inside said modular incubator chamber (300).

Clause 28. A modular incubator system (500) according to any of the clauses, wherein in respect of one or more of said one or more modular incubator chambers (300), said modular incubator chamber (300) comprises a chamber inlet opening for gas (312), wherein said chamber inlet opening for gas (312) is being in fluid connection with the interior (306) of said modular incubator chamber; and wherein said modular incubator chamber (300) furthermore comprises a chamber outlet opening for gas (314), wherein said chamber outlet opening for gas (314) is being in fluid connection with the interior (306) of said modular incubator chamber; and wherein in respect of one or more docking ports (402) of said docking station (400), said docking port (402) comprises a docking port outlet opening for gas (404) and a docking port inlet opening for gas (406); thereby enabling transfer of gas from said docking port (402) of said docking station (400) to the interior (306) of said modular incubator chamber (300) via said docking port outlet opening for gas (404) and said chamber inlet opening for gas (312); and thereby enabling transfer of gas from the interior (306) of said modular incubator chamber (300) to said docking port (402) of said docking station (400) via said chamber outlet opening for gas (314) and said docking port inlet opening for gas (406).

Clause 29. A modular incubator system (500) according to clause 28, wherein in respect of one or more of said one or more modular incubator chambers (300), and in respect of one or more of said one or more docking ports (402) of said docking station 400, the position of said chamber inlet opening for gas (312) of said housing (302) of said modular incubator chamber (300) and the position of said docking port outlet opening for gas (404) of said docking port (402) are adapted to each other in such a way that once docking said modular incubator chamber (300) in said docking port (402), said chamber inlet opening for gas (312) of said housing (302) of said modular incubator chamber (300) and said docking port outlet opening for gas (404) of said docking port (402) will be in fluid connection, thereby enabling transfer of gas from said docking port (402) to said modular incubator chamber (300); and
wherein the position of said chamber outlet opening for gas (314) of said housing (302) of said modular incubator chamber (300) and the position of said docking port inlet opening for gas (406) of said docking port (402) are adapted to each other in such a way that once docking said modular incubator chamber (300) in said docking port (402), said chamber outlet opening for gas (314) of said housing (302) of said modular incubator chamber (300) and said docking port inlet opening for gas (406) of said docking port (402) will be in fluid connection, thereby enabling transfer of gas from said modular incubator chamber (300) to said docking port (402).

Clause 30. A modular incubator system (500) according to any of the clauses 28 or 29, wherein said docking port outlet opening for gas (404) of said docking port (402) comprises a valve (4) and wherein said chamber inlet opening for gas (312) of said housing (302) comprises a valve (2); and wherein said chamber outlet opening for gas (314) comprises a valve (2) and wherein said docking port inlet opening for gas (406) of said docking port (402) comprises a valve (4).

Clause 31. A modular incubator system (500) according to any of the clauses 28-30; wherein in respect of one or more of said one or more modular incubator chambers (300), said valve (2) of said chamber inlet opening for gas (312) and said valve (2) of said chamber outlet opening for gas (314) each comprises a valve body (6) having a front end (10), a rear end (12) and a through-going channel (14) therein, and a spring-loaded displaceable valve element (8), wherein said displaceable valve element (8) is being arranged in said through-going channel (14); wherein said displaceable valve element (8) is being configured to be displaceable in said through-going channel (14) of said valve body (6) in such a way, that when not acted upon by an external force, said spring-loaded displaceable valve element (8) is not being displaced in said through-going channel (14) of said valve body (6), thereby making said valve attain a closed configuration blocking passage of gas through said through-going channel (14), and in such a way, that when acted upon by an external force, said spring-loaded displaceable valve element (8) is being displaced in said through-going channel (14) of said valve body (6), thereby making said valve (2) attain an open configuration, allowing passage of gas through said through-going channel (14); and
wherein in respect of one or more of said one or more docking ports (402) of said docking station (400), said valve (4) of said docking port outlet opening for gas (404) and said valve (4) of said docking port inlet opening for gas (406) each comprises a valve body (16) having a front end (20), a rear end (22) and a through-going channel (24) therein, and a spring-loaded displaceable valve element (18), wherein said displaceable valve element (18) is being arranged in said through-going channel (24); wherein said displaceable valve element (18) is being configured to be displaceable in said through-going channel (24) of said valve body (16) in such a way, that when not acted upon by an external force, said spring-loaded displaceable valve element (18) is not being displaced in said through-going channel (24) of said valve body (16), thereby making said valve attain a closed configuration blocking passage of gas through said through-going channel (24), and in such a way, that when acted upon by an external force, said spring-loaded displaceable valve element (18) is being displaced in said through-going channel (24) of said valve body (16), thereby making said valve (4) attain an open configuration, allowing passage of gas through said through-going channel (24).

Clause 32. A modular incubator system (500) according to any of the clauses 28-31, wherein in respect of one or more of said one or more docking ports (402) of said docking station (400), and in respect of one or more of said one or more modular incubator chambers (300) said valves (2,4) are having dimensions and geometries in such a way that once docking said modular incubator chamber (300) in said docking port (402) of said docking station (400), said displaceable valve element (8) of said valve (2) and said displaceable valve element (18) of said valve (4) will displace each other into their respective valve bodies (6,16), thereby opening said valves (2,4) of said docking port outlet opening for gas (404) and said chamber inlet opening for gas (312); and thereby opening said valves (2,4) of said chamber outlet opening for gas (314) and said docking port inlet opening for gas (406).

Clause 33. A modular incubator system (500) according according to any of the clauses 28-32, wherein in respect of one or more of said docking ports (402) of said docking station (400) of said modular incubator system (500), preferably in respect of all said docking ports (402), said docking port outlet opening for gas (404) comprises a flow restrictor for restricting the magnitude of flow of gas flowing into said docking port (402).

Clause 34. A modular incubator system (500) according to clause 33, wherein said flow restrictor comprises a tube through which the gas is conveyed to said docking port (402), wherein said tube optionally is having a cross-sectional area selected from the ranges of 0.2-8 mm², such as 0.5-7 mm², for example 1-6 mm², such as 2-5 mm² or 3-4 mm²; and/or wherein the length of said tube optionally is selected from the ranges of 5-30 mm, such as 8-25 mm, for example 10-22 mm, e.g. 15-20 mm.

Clause 35. A modular incubator system (500) according to any of the clauses 28-34, wherein said docking station (400) comprises a gas distribution system (204) for supplying gas to and from one or more of said one or more docking ports (402), wherein said gas distribution system (204) comprises a main gas supply line (210) and a main gas return line (212), wherein in respect of one or more of said docking ports (402), said docking port inlet opening for gas (404) is being fluidly connected to said main gas supply line (210), and said docking port outlet opening for gas (406) is being fluidly connected to said main gas return line (212).

Clause 36. A modular incubator system (500) according to clause 35 wherein said gas distribution system (204) comprises a number of manifold pairs (214), wherein each manifold pair comprises an inlet manifold (216) and an outlet manifold (218), wherein said inlet manifold (216) is being fluidly connected to said main gas supply line (210) and wherein said outlet manifold (218) is being fluidly connected to said main gas return line (212); wherein each manifold pair (214) is connected to one or more docking ports (402) of said docking station (400) in such a way that in respect of a specific manifold pair (214), and in respect of said one or more docking ports (402) being connected thereto, said docking port outlet opening for gas (404) of said docking port (402) is being fluidly connected to said inlet manifold (216), and said docking port inlet opening for gas (406) of said docking port (402) is being fluidly connected to said outlet manifold (218).

Clause 37. A modular incubator system (500) according to clause 35 or 36, wherein said docking station (400) comprises a gas supply system (200), wherein said gas supply system (200) comprises a gas source (202) and said gas distribution system (204), wherein said gas source comprises a supply gas outlet (206) and a return gas inlet (208), wherein said supply gas outlet (206) of said gas source (202) is being fluidly connected to said main gas supply line (210) of said gas distribution system (204), and wherein said return gas inlet (208) of said gas source (202) is being fluidly connected to said main gas return line (212) of said gas distribution system (204).

Clause 38. A modular incubator system (500) according to any of the clauses 35-37, wherein said gas source (202) of said gas supply system (200) comprises a gas mixing box (242) comprising said supply gas outlet (206) and said return gas inlet (208) of said gas source, wherein said main gas supply line (210) of said gas distribution system (204) is being fluidly connected to said supply gas outlet (206), and wherein said main gas return line (212) of said gas distribution system (204) is being fluidly connected to said return gas inlet (208) of said gas source (202), thereby forming a flow loop (244) comprising said gas distribution system (204) and said gas mixing box (242); wherein said flow loop comprises a pump (246) for circulating gas in said loop.

Clause 39. A modular incubator system (500) according to clause 38, wherein said pump (246) is being arranged downstream in relation to said main gas return line (212).

Clause 40. A modular incubator system (500) according to clause 38 or 40, wherein said flow loop (244) comprises a pump oscillation damper (247), wherein said pump oscillation damper optionally is being arranged immediately downstream in relation to said pump (246).

Clause 41. A modular incubator system (500) according to any of the clauses 38-40, wherein said flow loop (244) comprises a pressure sensor (248), such as a differential pressure sensor for sensing the pressure of gas supplied to said main gas supply line (210) of said gas distribution system (204), wherein said pressure senor (248) optionally is being arranged immediately upstream in relation to said main gas supply line (210) of said gas distribution system (204).

Clause 42. A modular incubator system (500) according to clause 41, wherein said pressure sensor (248) is being a differential pressure sensor, sensing a pressure relative to the pressure of the return gas inlet (208).

Clause 43. A modular incubator system (500) according to any of the clauses 38-41, wherein said flow loop (244) comprises a release valve (249) for enabling pressure relief in said flow loop, wherein said release valve optionally is being arranged immediately downstream in relation to said main gas return line (212) of said gas distribution system (402).

Clause 44. A modular incubator system (500) according to any of the clauses 38-43, wherein said gas mixing box (242) comprises an inlet for N₂ gas (250); and an inlet for CO₂ gas (251), wherein said inlet for N₂ gas (250) is fluidly connected to an N₂ valve (252) for regulating the inflow of N₂, and an N₂ mass flow sensor (253) arranged downstream of said N₂ valve (252) for sensing the amount of N₂ flowing into said gas mixing box (242); and wherein said inlet for CO₂ gas (251) is fluidly connected to a CO₂ valve (254) for regulating the inflow of CO₂, and an CO₂ mass flow sensor (255) arranged downstream of said CO₂ valve (254) for sensing the amount of CO₂ flowing into said gas mixing box (242).

Clause 45. A modular incubator system (500) according to any of the clauses 38-44, wherein said flow loop (244) comprises a mass flow sensor (256) arranged at an upstream position in relation to said gas mixing box (242) for sensing the amount of return gas entering said gas mixing box.

Clause 46. A modular incubator system (500) according to any of the clauses 38-45, wherein said gas source (202) comprises an O₂ sensor (258) for sensing the concentration of O₂ exiting said gas distribution system (204); and wherein said gas source (202) comprises a CO₂ sensor (260) for sensing the concentration of CO₂ exiting said gas distribution system (204), wherein said O₂ sensor and/or said CO₂ sensor optionally is/are being arranged downstream in relation to said pump (246).

Clause 47. A modular incubator system (500) according to any of the clauses 38-46, wherein said gas source (202) comprises a temperature sensor (262) for sensing the temperature of gas circulating in said flow loop (244), wherein said temperature sensor optionally is being arranged downstream in relation to said pump (246), preferably at a position corresponding to the position of said O₂ sensor (258).

Clause 48. A modular incubator system (500) according to any of the clauses 38-47, wherein said gas source (202) comprises a pressure sensor (264) for sensing the absolute pressure in said flow loop (244) wherein said pressure sensor optionally is being arranged downstream in relation to said pump (246), preferably at a position corresponding to the position of said CO₂ sensor (260).

Clause 49. A modular incubator system (500) according to any of the clauses 38-48, wherein said flow loop (244) comprises a UV sanitizer (266) for sanitizing gas flowing in said flow loop (244) via electromagnetic radiation in the UV range, wherein said UV sanitizer optionally being arranged immediately downstream in relation to said main gas return line (212).

Clause 50. A modular incubator system (500) according to any of the clauses 38-49, wherein said gas source (202) comprises one or more filters (268), such as HEPA and/or VOCs filters, wherein such a filter is being arranged immediately upstream in relation to said main gas supply line (210), and/or wherein such a filter is being arranged immediately upstream in relation to the inlet for N₂ gas (250) into said gas mixing box (242); and/or wherein such a filter is being arranged immediately upstream in relation to the inlet for CO₂ gas (251) into said gas mixing box (242).

Clause 51. A modular incubator system (500) according to any of the clauses 38-50, wherein said gas source (202) comprises a gas mixing control system (270), wherein said gas mixing control system is electrically connected to one or more of the following sensors for receiving sensing signals therefrom: said N₂ mass flow sensor (253) for sensing the amount of N₂ flowing into said gas mixing box; said CO₂ mass flow sensor (255) for sensing the amount of CO₂ flowing into said gas mixing box; said mass flow sensor (256) for sensing the amount of return gas entering said gas mixing box; said O₂ sensor (258) for sensing the concentration of O₂ exiting said main gas return line (212) of said gas distribution system (204); said CO₂ sensor (260) for sensing the concentration of CO₂ exiting said main gas return line (212) of said gas distribution system (204); said temperature sensor (262) for sensing the temperature circulating in said flow loop (244); said pressure sensor (264) for sensing an absolute pressure in said flow loop (244), said pressure sensor (248) for sensing the pressure of gas supplied to said gas main gas supply line (210) of said distribution system (204).

Clause 52. A modular incubator system (500) according to clause 51, wherein said gas mixing control system (270) is electrically connected to one or more of the following elements for control thereof: said N₂ valve (252) for regulating the inflow of N₂ into said gas mixing box (242); said CO₂ valve (254) for regulating the inflow of CO₂ to said gas mixing box (242); said pump (246) for circulating gas in said flow loop (244); said release valve (249).

Clause 53. A modular incubator system (500) according to clause 51 or 52, wherein said gas mixing control system (270) is being configured to receive input from said pressure sensor (248) and on the basis thereof control said pump (246), optionally also to activate said release valve (249) in order to maintain a desired and predetermined pressure of gas supplied to said main gas supply line (210) of said gas distribution system (204).

Clause 54. A modular incubator system (500) according to any of the clauses 51-53, wherein said gas mixing control system (270) is being configured to receive input from said mass flow sensor (256), and on the basis on said input to determine the total amount of CO₂ gas and N₂ gas needed to be supplied via said inlet for CO₂ gas (251) and via said inlet for N₂ gas (250) according to desired and predetermined criteria.

Clause 55. A modular incubator system (500) according to any of the clauses 51-54, wherein said gas mixing control system (270) is being configured to receive input from said CO₂ sensor (260) and said O₂ sensor (258), and on the basis of the CO₂ concentration sensed, is configured to control said CO₂ valve (254), by transmitting a control signal thereto, and thereby regulating the inflow of CO₂ gas in order to reach a desired and predetermined CO₂ concentration, and wherein subsequently, said gas mixing control system (270) on the basis of the O₂ concentration sensed, is configured to control said N₂ valve (252), by transmitting a control signal thereto, and thereby regulating the inflow of N₂ gas in order to reach a desired and predetermined O₂ concentration.

Clause 56. A modular incubator system (500) according to any of the clauses 51-55, wherein said gas mixing control system (270) is configured to use the input from said temperature sensor (262) for compensating the temperature sensitivity of said O₂ sensor (258).

Clause 57. A modular incubator system (500) according to any of the clauses 51-56, wherein said gas mixing control system (270) is configured to use the input from said pressure sensor (264) for compensating the pressure sensitivity of said CO₂ sensor (260).

Clause 58. A modular incubator system (500) according to any of the clauses 51-57, wherein said gas mixing control system (270) is being configured to maintain a pressure of gas supplied to said main gas supply line (210) of said gas distribution system (204), relative to the ambient atmospheric pressure, of 3-20 mbar, such as 5-18 mbar, such as 10-15 mbar above that ambient atmospheric pressure.

Clause 59. A modular incubator system (500) according to any of the clauses 51-58, wherein said gas mixing control system (270) is being configured to maintain a CO₂ concentration of gas entering said main gas supply line (210) of said gas distribution system (204) in the range of 5-10%, such as 6-9% or 7-8%; and/or an O₂ concentration of gas entering said main gas supply line 210 of said gas distribution system 204 in the range of 5-10%, such as 6-9% or 7-8%.

Clause 60. A modular incubator system (500) according to any of the preceding clauses, wherein said modular incubator system (500) comprises a control unit (650) for controlling the operation of said modular incubator system (500).

Clause 61. A modular incubator system (500) according to clause 60, wherein said control unit (650) is being coupled to an input device (652), such as an alphanumerical input device for allowing a user to provide settings input relating to a desired operational protocol of said modular incubator system.

Clause 62. A modular incubator system (500) according to clause 60 or 61, wherein said control unit (650) is being coupled to a display unit (654) for displaying, to a user, information relating to settings and/or operational status of said modular incubator system (300).

Clause 63. A modular incubator system (500) according to any of the clauses 60-62, wherein in respect of one or more docking ports (402) of said docking station (400) said control unit (650) is being configured for independently controlling one or more of the following: the temperature in the interior (306) of said modular incubator chamber (300) by controlling said electric heating element (318), said thermostat (374) and/or said thermostatic circuit (376), provision of power to said electric power source (320); provision of signals to said display (324) of a modular incubator chamber (300) being docked in said docking port (402), switching on and off of said active light source (352) of a modular incubator chamber (300) being docked in said docking port (402), or adjusting light intensity emitted therefrom, said image capturing device (408) of a docking port (402), said displacement device (482) for displacing said image capturing device (408), said gas mixing control system (270); and said image processing unit (660).

Clause 64. A modular incubator system (500) according to any of the clauses 60-63, wherein said control unit (650) is being coupled to a data processing unit (656) and optionally also to a data storage (658) for aiding in handling information during controlling of said modular incubator system.

Clause 65. A modular incubator system (500) according to any of the clauses 60-64, wherein said control unit (650) is being configured for conducting automatic operation of said modular incubator system (500) by configuring said control unit (650) to independently control one or more of the following: the temperature in the interior (306) of said modular incubator chamber (300) by controlling said electric heating element (318), said thermostat (374) and/or said thermostatic circuit (376), provision of power to said electric power source (320); provision of signals to said display (324) of a modular incubator chamber (300) being docked in said docking port (402), switching on and off of said active light source (352) of a modular incubator chamber (300) being docked in said docking port (402), or adjusting light intensity emitted therefrom, said image capturing device (408) of a docking port (402), said displacement device (482) for displacing said image capturing device (408), said gas mixing control system (270); and said image processing unit (660).

Clause 66. A modular incubator system (500) according to any of the clauses 60-65, wherein said control unit (650) is being configured for enabling time lapse capturing of images by said image capturing devices (408).

### Clause 67. a Modular Incubator Chamber (300) Comprising:

a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber (300) and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300);
wherein said housing (302) of said modular incubator chamber (300) comprises a transparent window (316) for enabling capturing of images of a biological material M being accommodated in the interior thereof, through said transparent window (316).

Clause 68. A modular incubator chamber (300) according to clause 67, wherein said incubator chamber (300) is comprising features as defined in respect of the modular incubator chamber (300) of the modular incubator system (500) according to any of the clauses 1-66.

Clause 69. A docking station (400) for docking one or more modular incubator chambers (300); wherein said docking station comprises one or more docking ports (402) for receiving a housing (302) of one or more of said incubator chambers (300);
wherein in respect of one or more docking ports (402) of said docking station, said docking port comprises an image capturing device (408) for capturing an image of the interior (306) of a modular incubator chamber (300), once being docked in said docking port (402).

Clause 70. A docking station (400) according to clause 69, wherein said docking station (400) is comprising features as defined in respect of the docking station (400) of the modular incubator system (500) according to any of the clauses 1-66.

Clause 71. Use of a modular incubator system (500) according to any of the clauses 1-66, for incubating a viable biological material.

Clause 72. Use of a modular incubator chamber (300) according to any of the clauses 67 or 68, for incubating a viable biological material.

Clause 73. Use of a docking station (400) according to any of the clauses 69 or 70, for incubating a viable biological material.

Clause 74. Use according to any of the clauses 71-73, wherein said biological material is being an oocyte or an embryo, such as a human oocyte or a human embryo.

Clause 75. A method of incubating a viable biological material, wherein said method comprises:
i) providing a modular incubator system (500) according to any of the clauses 1-66;
ii) providing a viable biological material;
iii) arranging said viable biological material in a culture dish (310) and subsequently arranging said culture dish in the interior (306) of a modular incubator chamber (300) of said modular incubator system (400);
iv) docking said modular incubator chamber (300) in a docking port (402) of said docking station (400) of said incubator system (500);
v) allowing said viable biological material to be incubated in said modular incubator chamber (300);
vi) whenever desired allowing said image capturing device (408) to capture one or more images of said biological material being accommodated in said culture dish (310).

Clause 76. a method according to clause 75 further comprising the step of:
vii) removing said incubator chamber (300) from said docking port (402) of said docking station (400), when desired, in order to manually inspect the viable biological material, and optionally also to remove, add or exchange growth medium/media in said culture dish (310).

### LIST OF REFERENCE NUMERALS

2 Valve
4 Valve
6 First valve body of first valve
8 First valve element of first valve
10 Front end of first valve body
12 Rear end of first valve body
14 First throughgoing channel of first valve
16 Second valve body of second valve
18 Second valve element of second valve
20 Front end of second valve
22 Rear end of second valve
24 Second throughgoing channel of second valve
26 First spring of first valve
28 Second spring of second valve
100 Valve system
200 Gas supply system
202 Gas source of gas supply system
204 Gas distribution system of gas supply system
206 Supply gas outlet of gas source
208 Return gas inlet of gas source
210 Main gas supply line of gas distribution system
212 Main gas return line of gas distribution system
214 Manifold pair
216 Inlet manifold of manifold pair
218 Outlet manifold of manifold pair
228 Group of docking ports
242 Gas mixing box
244 Flow loop of gas supply system
246 Pump of gas source
247 Pump oscillation damper
248 Pressure sensor for sensing pressure of gas supplied to main gas supply line
249 Release valve
250 Inlet for N₂ gas
251 Inlet for CO₂ gas
252 N₂ valve
253 N₂ mass flow sensor
254 CO₂ valve
255 CO₂ mass flow sensor
256 Mass flow sensor for sensing the amount of return gas flowing into gas mixing box
258 O₂ sensor
260 CO₂ sensor
262 Temperature sensor
264 Pressure sensor
266 UV sanitizer
268 Filter
270 Gas mixing control system
300 Modular incubator chamber
302 Housing of modular incubator chamber
304 Lid of modular incubator chamber
306 Interior of modular incubator chamber
308 Culture dish support
310 Culture dish
312 Modular incubator chamber inlet opening for gas
314 Modular incubator chamber outlet opening for gas
316 Transparent window of housing of modular incubator chamber
318 Electric heating element
320 Electric power source
322 Electric connectors of modular incubator chamber
324 Display of housing of modular incubator chamber
326 Incubation chamber engagement means of modular incubator chamber
340 First end of modular incubator chamber
342 Second end of modular incubator chamber
358 Bottom part of modular incubator chamber
372 Light source
374 Thermostat
376 Thermostatic circuit
400 Docking station
402 Docking port of docking station
404 Docking port outlet opening for gas
406 Docking port inlet opening for gas
408 Image capturing device of docking port of docking station
410 Electric connector of docking port
414 Docking port engagement means of docking port of docking station
482 Displacement device for displacing image capturing unit
500 Modular incubator system
650 Control unit
652 Input device
654 Display unit
656 Data processing unit
658 Data storage
660 Image processing unit
X Longitudinal direction of modular incubator chamber
Y Transversal direction perpendicular to longitudinal direction X

1.-76. (canceled)

77. A modular incubator system (500) for incubating a viable biological material M, said modular incubator system comprising:
one or more modular incubator chambers (300) in combination with
a docking station (400);
wherein in respect of one or more of said one or more modular incubator chambers (300), said modular incubator chamber (300) comprises a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300); wherein said housing (302) of said modular incubator chamber (300) comprises a transparent window (316) for enabling capturing of images of a biological material M being accommodated in the interior thereof, through said transparent window;
wherein in respect of one or more of said modular incubator chambers (300), said modular incubator chamber comprises in its interior (306) an electric heating element (318) for heating the interior of said modular incubator chamber, and wherein said modular incubator chamber comprises a power source (320) for providing power to said heating element (318), wherein said electric heating element (318) is being electrically connected to said power source (320);
wherein the individual incubation chamber is configured in a way that enables support on a planar, horizontal support surface, thereby making the incubator chambers configured to be used for incubation of a viable biological material, irrespective of whether the individual incubator chamber is being docked in a docking port of the docking station, or whether that incubator chamber is removed from the docking port of the docking station;
wherein said docking station (400) comprises one or more docking ports (402) for receiving a housing (302) of one or more of said incubator chambers (300);
wherein in respect of one or more docking ports (402) of said docking station (400), said docking port comprises an image capturing device (408) for capturing an image of the interior (306) of a modular incubator chamber (300), once being docked in said docking port (402).

78. A modular incubator system (500) according to claim 77, wherein in respect of one or more of said one or more modular incubator chambers (300) and in respect of one or more of said one or more docking ports (402) of said docking station (400), the position of said transparent window (316) of said modular incubator chamber (300) is adapted to the position of said image capturing device (408) in said docking port (402) in a way that enables capturing of images by said image capturing device (408) through said transparent window (316) of said modular incubator chamber (300), once said modular incubator chamber (300) is being docked in said docking port (402).

79. A modular incubator system (500) according to claim 77, wherein in respect of one or more of said one or more modular incubator chambers (300), said modular incubator chamber (300), in the interior (306) thereof, comprises a light source (372) for directing light to the area of the culture dish support (308) of said modular incubator chamber (300), thereby enabling illumination of a viable biological material in a situation of capturing images of said viable biological material.

80. A modular incubator system (500) according to claim 77, wherein said modular incubator system (500) comprises an image processing unit (660) for image processing of images captured by said image capturing device (408), wherein said modular incubator system (400) optionally furthermore comprises a data storage (658) for storing images captured by said image capturing units (408) and/or for storing images processed by said image processing unit (660).

81. A modular incubator system (500) according to claim 80, wherein one or more of said image capturing devices (408) of said docking ports (402) of said docking station is/are being coupled to an image processing unit (660).

82. A modular incubator system (500) according to claim 77, wherein in respect of one or more specific docking ports (402) of said docking station (400), said specific docking port comprises its own dedicated image capturing device (408) which is configured to only capture images relating to a modular incubator chamber (300) which is being docked in said specific docking port (402).

83. A modular incubator system (500) according to claim 77, wherein in respect of a number N of adjacently arranged docking ports (402) of said docking station (400), said adjacently arranged docking ports share a common image capturing device (408) in the sense that one and only one image capturing device is responsible for capturing images relating to a modular incubator chamber (300) which is being docked in one of said N adjacently arranged docking ports (402), wherein said docking station comprises a displacement device (482) for enabling displacement of said common image capturing device (408) in relation to said N adjacently arranged docking ports (402) of said docking station (400).

84. A modular incubator system (500) according to claim 77, wherein in respect of one or more of said one or more modular incubator chambers (300), said modular incubator chamber (300) comprises a chamber inlet opening for gas (312), wherein said chamber inlet opening for gas (312) is being in fluid connection with the interior (306) of said modular incubator chamber; and wherein said modular incubator chamber (300) furthermore comprises a chamber outlet opening for gas (314), wherein said chamber outlet opening for gas (314) is being in fluid connection with the interior (306) of said modular incubator chamber; and wherein in respect of one or more docking ports (402) of said docking station (400), said docking port (402) comprises a docking port outlet opening for gas (404) and a docking port inlet opening for gas (406); thereby enabling transfer of gas from said docking port (402) of said docking station (400) to the interior (306) of said modular incubator chamber (300) via said docking port outlet opening for gas (404) and said chamber inlet opening for gas (312); and thereby enabling transfer of gas from the interior (306) of said modular incubator chamber (300) to said docking port (402) of said docking station (400) via said chamber outlet opening for gas (314) and said docking port inlet opening for gas (406).

85. A modular incubator system (500) according to claim 84, wherein said docking port outlet opening for gas (404) of said docking port (402) comprises a valve (4) and wherein said chamber inlet opening for gas (312) of said housing (302) comprises a valve (2); and wherein said chamber outlet opening for gas (314) comprises a valve (2) and wherein said docking port inlet opening for gas (406) of said docking port (402) comprises a valve (4).

86. A modular incubator system (500) according to claim 84, wherein said docking station (400) comprises a gas distribution system (204) for supplying gas to and from one or more of said one or more docking ports (402), wherein said gas distribution system (204) comprises a main gas supply line (210) and a main gas return line (212), wherein in respect of one or more of said docking ports (402), said docking port inlet opening for gas (404) is being fluidly connected to said main gas supply line (210), and said docking port outlet opening for gas (406) is being fluidly connected to said main gas return line (212).

87. A modular incubator system (500) according to claim 86 wherein said gas distribution system (204) comprises a number of manifold pairs (214), wherein each manifold pair comprises an inlet manifold (216) and an outlet manifold (218), wherein said inlet manifold (216) is being fluidly connected to said main gas supply line (210) and wherein said outlet manifold (218) is being fluidly connected to said main gas return line (212); wherein each manifold pair (214) is connected to one or more docking ports (402) of said docking station (400) in such a way that in respect of a specific manifold pair (214), and in respect of said one or more docking ports (402) being connected thereto, said docking port outlet opening for gas (404) of said docking port (402) is being fluidly connected to said inlet manifold (216), and said docking port inlet opening for gas (406) of said docking port (402) is being fluidly connected to said outlet manifold (218).

88. A modular incubator system (500) according to claim 87, wherein said docking station (400) comprises a gas supply system (200), wherein said gas supply system (200) comprises a gas source (202) and said gas distribution system (204), wherein said gas source comprises a supply gas outlet (206) and a return gas inlet (208), wherein said supply gas outlet (206) of said gas source (202) is being fluidly connected to said main gas supply line (210) of said gas distribution system (204), and wherein said return gas inlet (208) of said gas source (202) is being fluidly connected to said main gas return line (212) of said gas distribution system (204).

89. A modular incubator system (500) according to claim 77, wherein said modular incubator system (500) comprises a control unit (650) for controlling the operation of said modular incubator system (500).

90. A modular incubator system (500) according to claim 89, wherein said control unit (650) is being coupled to a data processing unit (656) and optionally also to a data storage (658) for aiding in handling information during controlling of said modular incubator system.

91. A modular incubator system (500) according to claim 89, wherein said control unit (650) is being configured for enabling time lapse capturing of images by said image capturing devices (408).

92. Use of a modular incubator system (500) according to claim 77 for incubating a viable biological material.

93. A modular incubator chamber (300) comprising:
a housing (302) having a first end (340) and a second end (342), thereby defining a longitudinal direction X between said first end and said second end;
wherein said housing comprises a lid (304), wherein said lid is being configured to be able to shift between an open configuration allowing access to the interior (306) of said modular incubator chamber (300) and a closed configuration, sealing off access to the interior of said modular incubator chamber;
wherein said modular incubator chamber (300), at said interior (306) thereof, comprises a culture dish support (308) for positioning a culture dish (310) with the view to accommodate one or more biological materials M within the housing (302) of said modular incubator chamber (300);
wherein said housing (302) of said modular incubator chamber (300) comprises a transparent window (316) for enabling capturing of images of a biological material M being accommodated in the interior thereof, through said transparent window (316).

94. Use of a modular incubator chamber (300) according to claim 93, for incubating a viable biological material.

95. Use according to claim 94, wherein said biological material is being an oocyte or an embryo, such as a human oocyte or a human embryo.

96. A method of incubating a viable biological material, wherein said method comprises:
i) providing a modular incubator system (500) according to claim 77;
ii) providing a viable biological material;
iii) arranging said viable biological material in a culture dish (310) and subsequently arranging said culture dish in the interior (306) of a modular incubator chamber (300) of said modular incubator system (400);
iv) docking said modular incubator chamber (300) in a docking port (402) of said docking station (400) of said incubator system (500);
v) allowing said viable biological material to be incubated in said modular incubator chamber (300);
vi) whenever desired allowing said image capturing device (408) to capture one or more images of said biological material being accommodated in said culture dish (310).
